(19) United States
(12) Patent Application Publication
(10) Pub. No.: US 2023/0063297 A1
(43) Pub. Date: Mar. 2, 2023

(54) HAIR TREATMENT COMPOSITIONS PROVIDING BRIGHTENING AND SHINE

(71) Applicant: L’OREAL, Paris (FR)

(72) Inventors: Kazumitsu KAWAKAMI, Westfield, NJ (US); Liliana XAVIER, Mountainside, NJ (US); Anna BOTTO, Cranford, NJ (US)

(21) Appl. No.: 17/401,671

(22) Filed: Aug. 13, 2021

(51) Int. Cl. A61K 8/67; A61K 8/44; A61K 8/46; A61K 8/41; A61K 8/92; A61K 8/73; A61K 8/34; A61K 8/898; A61Q 5/02; A61Q 5/12; A61Q 5/06

(52) U.S. Cl. CPC A61K 8/676; A61K 8/44; A61K 8/463; A61K 8/416; A61K 8/922; A61K 8/737; A61K 8/342; A61K 8/345; A61K 8/898; A61Q 5/02; A61Q 5/12; A61Q 5/065; A61K 2800/43; A61K 2800/51; A61K 2800/5426; A61K 2800/596; A61K 2800/805; A61K 2800/884

(57) ABSTRACT

The instant disclosure relates to hair treatment compositions that provide brightening, shine, luster, and cleanliness and/or conditioning to hair, wherein the hair treatment compositions include: (a) ascorbyl glucoside; (b) an amino acid chelant; (c) N,N-dicarboxymethyl glutamic acid, a salt thereof, and/or a combination thereof; (d) one or more surfactants; and (e) water. Also disclosed are methods for using the hair treatment compositions, for example, for enhancing brightness, shine, luster, and cleanliness and/or conditioning of the hair.

### FIELD OF THE DISCLOSURE

The instant disclosure relates to hair treatment compositions that provide brightening, shine, luster, and cleanliness and/or conditioning to hair. Also disclosed are methods for using the hair treatment compositions, for example, for enhancing brightness, shine, luster, and cleanliness and/or conditioning to hair.

### BACKGROUND

Many consumers desire to use cosmetic and care compositions that enhance the appearance of hair, e.g., by changing the color, style, and/or shape of the hair, and/or by imparting various cosmetic properties to hair, such as shine and brightness. A common problem associated with obtaining and maintaining lustrous hair are inadequate for enhancing brightness and shine, especially for bleach bleached and/or colored hair, or hair subjected to other chemical treatments.

The process of changing the color of hair, for example, can involve depositing an artificial color onto the hair which provides a different shade or color to the hair, and/or lifting the color of the hair (bleaching the hair), such as lightening the color of dark hair to lighter shades using oxidizing agents. Additionally, there are many techniques and compositions for styling or altering the shape of hair. For example, hair care products referred to as “hair relaxers” or “hair straighteners” can relax or straighten wavy, curly, or kinky hair. Different types of compositions can be applied onto hair to change its shape and make it more manageable, such as alkaline and acidic compositions. Hair coloring, bleaching, relaxing, straightening, curling, or waving treatments may be applied in a hair salon by a professional or in the home by the individual consumer.

While dyeing or color lifting compositions can effectively alter the color of hair, and chemical treatments can effectively alter the shape of the hair, these chemical processes can damage hair fibers making them more susceptible to metal ions present in tap water, ocean water, swimming pools, etc. For example, “hard water” (untreated tap water) often includes high amounts of dissolved mineral salts, such as calcium and magnesium salts. Copper, iron, and chlorine salts are also prevalent. Chlorine is a particularly problematic compound in swimming pool water. It is difficult to completely rinse out mineral content from the hair, which tends to build-up over time. The same chemical compounds in water that cause a whitish build up (scum) on bathtubs and shower curtains, deposits on the surface of hair. Subsequent exposure to water during washing and conditioning results in additional build-up.

Mineral salt deposits on hair interfere with cleansing agents in shampoos making them less effective. It takes longer to create a lather, so larger quantities of the shampoo are needed, even if the shampoo contains strong cleansing agents. Build-up of mineral salts in hair causes a “dulling effect.” The hair losses shine, brightness, and luster. Over time, the build-up even influences the color of hair, especially artificially lightened (bleached) and/or colored hair, and negatively impacts the ability to easily style the hair.

Consumers seek products that prevent, eliminate, and/and mitigate the dulling effect mineral salts impart to hair. Hair should have a natural, healthy, and light weight feel, and maintain a vibrant color. Further, consumers seek simple products that offer multiple benefits without requiring time consuming and complicated procedures. The object of the instant disclosure is to provide hair treatment compositions and methods for treating hair that fulfill these requirements.

### SUMMARY OF THE DISCLOSURE

The hair treatment compositions of the instant disclosure provide surprising brightening, shine, luster, and cleanliness and/or conditioning to hair. The inventors discovered that ascorbyl glucoside in conjunction with an amino acid chelant and N,N-dicarboxymethyl glutamic acid (GLDA), a salt thereof, and/or a combination thereof is surprisingly effective for eliminating, minimizing, and/or preventing unwanted build-up on hair caused, for example, by mineral salts (and ions of mineral salts) prevalent in water. Without wishing to be bound by any particular theory, the inventors believe that a combination of ascorbyl glucoside, an amino acid chelant, and N,N-dicarboxymethyl glutamic acid, a salt thereof, and/or a combination thereof sequesters the mineral salts (or ions of the mineral salts) allowing them to be easily removed from the hair, even when the mineral salts (or ions of the mineral salts) have been absorbed into the hair. In addition, the combination helps prevent mineral salts (or ions of the mineral salt) from being deposited onto/into the hair from the beginning, eliminating the need for subsequent removal. Thus, the hair treatment compositions improve the brightness, shine, and luster of the hair, and prevent unwanted color changes. Hair treated with the compositions is vibrant, lightweight, and natural looking.

The hair treatment compositions typically include:
(a) ascorbyl glucoside;
(b) an amino acid chelant;
(c) N,N-dicarboxymethyl glutamic acid, a salt thereof, and/or a combination thereof (GLDA);
(d) one or more surfactants; and
(e) water.

Nonlimiting examples of useful amino acid chelants include glycine, lysine, methionine, and combinations thereof. Nonlimiting examples of a salt of N,N-dicarboxymethyl glutamic acid is N,N-Dicarboxymethyl glutamic acid tetrasodium salt.

Surfactants are included, for example, to assist with cleansing and conditioning the hair. Anionic surfactants are useful for their cleansing attributes. Anionic surfactants have a negative charge on their hydrophilic end. The negative charge helps the surfactant molecules lift and suspend soils in micelles. The anionic surfactants also assist in the removal of minerals sequestered by the combination of ascorbyl glucoside and chelants.

Cationic surfactants have a positive charge on their hydrophilic end. Rather than being used primarily for detergency, cationic surfactants provide conditioning properties. The surface of hair typically has an overall negative charge, which becomes more prevalent if the cuticle is damaged. The head groups of cationic surfactants experience electrostatic attraction to the negative charge of the hair. The hydrophobic portion of the surfactant smooths the cuticle, reduces static, and helps prevent tangle formation.

In some instances, the hair treatment compositions may include one or more cationic conditioning polymers. As the name suggests, cationic conditioning polymers are useful for providing conditioning properties to the hair. Cationic conditioning polymers help impart smoothing to the hair surface and help improve combability of the hair.

In some instances, the hair treatment compositions may include water-soluble solvents, in particular organic solvents. Nonlimiting examples include glycerin, mono-alcohols, polyols (polyhydric alcohols), glycols, and a mixture thereof. Water-soluble solvents tend be multifunctional. For example, they can help solubilize ingredients in a composition, impart antimicrobial properties to a composition, boost effectiveness, improve spreadability or “feel” of a product, etc. Water is also included in the hair treatment compositions.

The hair treatment composition can be formulated as a shampoo, a conditioner, a conditioning shampoo, or as a standalone product, for example, a product used separately or in conjunction with a shampoo, conditioner, or other product. When the hair treatment composition is a shampoo, it will typically include anionic surfactants. When the hair treatment composition is a conditioner, it will typically include cationic surfactants. The surfactants useable in the hair treatment compositions, however, are not limited to anionic and cationic surfactants. The hair treatment compositions will often include nonionic, amphoteric, and/or zwitterionic surfactants.

The hair treatment compositions may be included in a kit, for example, a kit comprising a hair treatment composition according to the instant disclosure and one or more separately contained compositions. For example, the kit may include a shampoo according to the instant disclosure, and one or more separately contained compositions, for example a conditioner. The separately contained conditioner may be a conditioner according to the instant disclosure or may be an independent conditioner. Similarly, the kit may include a conditioner according to the instant disclosure, and one or more separately contained compositions, for example a shampoo. The separately contained shampoo may be a shampoo according to the instant disclosure or may be an independent shampoo.

Another aspect of the instant disclosure relates to methods for treating hair using the hair treatment compositions. The hair treatment compositions are particularly useful in a “rinse off” treatment where the compositions are applied to the hair, optionally allowed to remain on the hair for a period, and subsequently rinsed from the hair. Rinsing the compositions from hair allows for the mineral complexes to be washed/rinsed away from the hair. The hair treatment compositions are typically applied to wet or damp hair but may be applied to dry hair. The hair treatment compositions are massaged or lathered into the hair before rinsing.

If the hair treatment composition is a shampoo, it can be used to cleanse the hair while simultaneously acting on mineral salts (or ions of mineral salts) to dislodge/remove them or prevent them from attaching to the hair. If the hair treatment composition is a conditioner it can be used to condition the hair (e.g., provide smoothing, static reduction, and/or tangle reduction) while simultaneously acting on mineral salts (or ions of mineral salts) to dislodge/remove them or prevent them from attaching to the hair. In instances where the hair treatment composition is not necessarily a shampoo or a conditioner, the composition can be applied to the hair, optionally allowed to remain on the hair for a period, and subsequently rinsed from the hair. The treated hair may be artificially colored hair (bleached and/or colored hair), chemically treated hair (such as chemically straightened or curled hair), or natural hair (hair that has not been artificially colored or otherwise chemically treated). The methods are particularly useful for imparting shine, luster, vibrance, and brightening to hair; and for preventing/reducing color changes from hair, especially artificially colored hair (bleached and/or colored hair) and blonde or naturally light hair.

### DETAILED DESCRIPTION OF THE DISCLOSURE

The term “hair treatment composition” encompasses many types of compositions for application to the hair, for example, hair lotions, hair emulsion creams, hair gel creams, hair conditioners, hair masques (masks), shampoos, conditioners, hair rinses, etc., which can be used either as leave-on or rinse-off treatments or products. The hair treatment compositions are characterized by their ability to provide shine, luster, vibrance, and brightening to hair.

The hair treatment compositions include: (a) ascorbyl glucoside; (b) an amino acid chelant; (c) N,N-dicarboxymethyl glutamic acid, a salt thereof, and/or a combination thereof; (d) one or more surfactants; and (e) water. The amounts for these components may vary, but typically, the hair treatment compositions include:

(a) about 0.1 to about 5 wt. % of the ascorbyl glucoside;

(b) about 0.01 to about 3 wt. % of the amino acid chelant;

(c) about 0.01 to about 3 wt. % of N,N-dicarboxymethyl glutamic acid, a salt thereof, and/or a combination thereof;
(d) about 0.5 to about 25 wt. % of the one or more surfactants; and
(e) about 50 to about 95 wt. % of the water;
   wherein all weight percentages are based on the total weight of the hair treatment composition.

The amount of ascorbyl glucoside can vary but the hair treatment compositions typically include about 0.1 to about 5 wt. % ascorbyl glucoside, based on the total weight of the hair treatment composition. In some instances, the hair treatment compositions includes about 0.1 to about 4 wt. %, about 0.1 to about 3 wt. %, about 0.1 to about 2 wt. %, about 0.2 to about 5 wt. %, about 0.2 to about 4 wt. %, about 0.2 to about 3 wt. %, about 0.2 to about 2 wt. %, about 0.3 to about 5 wt. %, about 0.3 to about 4 wt. %, about 0.3 to about 3 wt. %, about 0.3 to about 2 wt. %, including ranges and subranges therebetween, based on the total weight of the hair treatment composition.

Nonlimiting examples of amino acid chelants that are useful in the hair treatment compositions include glycine, lysine, methionine, and combinations thereof. In some instances, the amino acid chelant is glycine. The total amount of the amino acid chelant may vary but is typically about 0.01 to about 3 wt. %, based on the total weight of the hair treatment composition. In some instances, the total amount of the amino acid chelants in the hair treatment compositions is about 0.01 to about 2 wt. %, about 0.01 to about 1 wt. %, about 0.05 to about 3 wt. %, about 0.05 to about 2 wt. %, about 0.05 to about 1 wt. %, about 0.1 to about 3 wt. %, about 0.1 to about 2 wt. %, about 0.1 to about 1 wt. %, including ranges and subranges therebetween, based on the total weight of the hair treatment composition.

A particularly useful d salt of N,N-dicarboxymethyl glutamic acid is tetrasodium glutamate diacetate. The total amount of N,N-dicarboxymethyl glutamic acid, a salt thereof, and/or a combination thereof may vary but is typically about 0.01 to about 3 wt. %, based on the total weight of the hair treatment composition. In some instances, the total amount of N,N-dicarboxymethyl glutamic acid, a salt thereof, and/or a combination thereof in the hair treatment compositions is about 0.01 to about 2 wt. %, about 0.01 to about 1 wt. %, about 0.05 to about 3 wt. %, about 0.05 to about 2 wt. %, about 0.05 to about 1 wt. %, about 0.1 to about 3 wt. %, about 0.1 to about 2 wt. %, about 0.1 to about 1 wt. %, including ranges and subranges therebetween, based on the total weight of the hair treatment composition.

The hair treatment compositions include one or more surfactants, which may be anionic, cationic, nonionic, and/or amphoteric/zwitterionic. Combinations of these types of surfactants are often useful. The amount and types of surfactants may vary depending on whether the composition is a shampoo, a conditioner, or another type of composition. Shampoos typically include one or more anionic surfactants. Conditioners typically include one or more cationic surfactants. Regardless, the hair treatment compositions may include from about 0.5 to about 25 wt. % of total surfactants (all types of surfactants), based on the total weight of the hair treatment composition. In some instances, the hair treatment compositions include about 0.5 to about 20 wt. %, about 0.5 to about 15 wt. %, about 0.5 to about 10 wt. %, about 0.5 to about 5 wt. %, about 1 to about 25 wt. %, about 1 to about 20 wt. %, about 1 to about 15 wt. %, about 1 to about 10 wt. %, about 1 to about 5 wt. %, about 5 to about 25 wt. %, about 5 to about 20 wt. %, or about 5 to about 15 wt. %, including ranges and subranges therebetween, based on the total weight of the hair treatment composition.

Nonlimiting examples of anionic surfactants include alkyl sulfates, alkyl ether sulfates, alkyl sulfonates, alkyl sulfosuccinates, alkyl sulfoacetates, acyl isethionates, alkoxylated monoacids, acyl amino acids such as acyl taurates, acyl glycinates, acyl glutamates, acyl sarcosinates, salts thereof, and a mixture thereof. Alkyl sulfates and alkyl ether sulfates have particularly strong cleansing attribute. Examples are sodium lauryl sulfate (SLS) or sodium dodecyl sulfate (SDS). In some instances, alky sulfates are particularly useful. A more exhaustive but nonlimiting list of useful anionic surfactants is included below, under the heading “Anionic Surfactants.”

Nonlimiting examples of cationic surfactants include include cetrimonium chloride, stearimonium chloride, behentrimonium chloride, behentrimonium methosulfate, behenam idopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidam ido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, and mixtures thereof. In some instances, cetrimonium chloride, behentrimonium chloride, and/or behentrimonium methosulfate are particularly useful. A more exhaustive but nonlimiting list of useful cationic surfactants is included below, under the heading “Cationic Surfactants.”

Nonlimiting examples of nonionic surfactants include alkyl polyglucosides and alcohols, alpha-diols, alkylphenols and esters of fatty acids, being ethoxylated, propoxylated or glycerolated, ethoxylated fatty esters, glyceryl esters of fatty acids, fatty alcohol ethoxylates, alkyl phenol ethoxylates, fatty acid alkoxylates, and mixtures thereof. A more exhaustive but nonlimiting list of useful nonionic surfactants is provided below, under the heading “Nonionic Surfactants.”

Nonlimiting examples of amphoteric surfactants include betaines, alkyl sultaines, alkyl amphoacetates, alkyl amphoproprionates, and mixtures thereof. In some instances, betaines are particularly useful. Nonlimiting examples of betaines include coco betaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, and mixtures thereof. Typically, at least one betaine compound is selected from coco betaine, behenyl betaine, capryl/capramidopropyl betaine, and lauryl betaine, and mixtures thereof. Particularly preferred betaines include coco betaine and cocamidopropyl betaine. A more exhaustive but nonlimiting list of useful amphoteric surfactants is provided below, under the heading “Amphoteric Surfactants.”

In some instances, the hair treatment composition is a shampoo including one or more anionic surfactants, for example, from about 0.5 to about 25 wt. % of one or more anionic surfactants based on the total weight of the hair treatment composition (the total weight of the shampoo). In some instances, the shampoo includes from about 0.5 to about 20 wt. %, about 0.5 to about 15 wt. %, about 1 to about 25 wt. %, about 1 to about 20 wt. %, about 1 to about 15 wt. %, about 5 to about 25 wt. %, about 5 to about 20 wt. %, about 5 to about 15 wt. %, about 8 to about 25 wt. %, about 8 to about 20 wt. %, or about 8 to about 15 wt. %, including ranges and subranges therebetween, based on the total weight of the hair shampoo.

In some instances, the hair treatment composition is a conditioner including one or more cationic surfactants, for example, from about 0.1 to about 15 wt. % of one or more cationic surfactants based on the total weight of the hair treatment compositions (the total weight of the conditioner). In some instances, the conditioner includes about 0.1 to about 10 wt. %, about 0.1 to about 8 wt. %, about 0.1 to about 5 wt. %, about 0.1 to about 3 wt. %, about 0.5 to about 15 wt. %, about 0.5 to about 10 wt. %, about 0.5 to about 8 wt. %, about 0.5 to about 5 wt. %, about 0.5 to about 3 wt. %, about 1 to about 15 wt. %, about 1 to about 10 wt. %, about 1 to about 8 wt. %, about 1 to about 5 wt. %, or about 1 to about 3 wt. %, including ranges and subranges therebetween, based on the total weight of the hair treatment composition.

The hair treatment compositions of the instant disclosure may include one or more nonionic surfactants, regardless of whether the composition is a shampoo, conditioner, or other type of composition. The total amount of nonionic surfactants may vary, but typically are in an amount of about 0.01 to about 15 wt. %, about 0.01 to about 10 wt. %, about 0.01 to about 5 wt. %, about 0.01 to about 3 wt. %, about 0.1 to about 15 wt. %, about 0.1 to about 10 wt. %, about 0.1 to about 5 wt. %, about 0.1 to about 3 wt. %, about 0.5 to about 15 wt. %, about 0.5 to about 10 wt. %, about 0.5 to about 5 wt. %, about 0.5 to about 3 wt. %, about 1 to about 15 wt. %, about 1 to about 10 wt. %, about 1 to about 5 wt. %, about 1 to about 3 wt. %, including ranges and subranges therebetween, based on the total weight of the hair treatment composition.

Amphoteric surfactants may also optionally be included in the hair treatment compositions, regardless of whether the hair treatment composition is a shampoo, conditioner, or other type of composition. The total amount of amphoteric surfactants may vary but typically are in an amount of about 0.01 to about 15 wt. %, about 0.01 to about 10 wt. %, about 0.01 to about 5 wt. %, about 0.01 to about 3 wt. %, about 0.1 to about 15 wt. %, about 0.1 to about 10 wt. %, about 0.1 to about 5 wt. %, about 0.1 to about 3 wt. %, about 0.5 to about 15 wt. %, about 0.5 to about 10 wt. %, about 0.5 to about 5 wt. %, about 0.5 to about 3 wt. %, about 1 to about 15 wt. %, about 1 to about 10 wt. %, about 1 to about 5 wt. %, about 1 to about 3 wt. %, including ranges and subranges therebetween, based on the total weight of the hair treatment composition.

The hair treatment compositions are aqueous composition. The total amount of water may vary but is typically in an amount of about 50 to about 95 wt. %, based on the total weight of the hair treatment composition. In some instances, the total amount of water is about 60 to about 95 wt. %, about 70 to about 95 wt. %, about 75 to about 95 wt. %, about 80 to about 95 wt. %, about 50 to about 92 wt. %, about 60 to about 92 wt. %, about 70 to about 92 wt. %, about 80 to about 92 wt. %, including ranges and subranges therebetween, based on the total weight of the hair treatment composition.

The hair treatment compositions may optionally include one or more water-soluble solvents. The term “water-soluble solvent” is interchangeable with the term “water-miscible solvent” and means a compound that is liquid at 25° C. and at atmospheric pressure (760 mmHg), and it has a solubility of at least 50% in water under these conditions. In some cases, the water-soluble solvent has a solubility of at least 60%, 70%, 80%, or 90%. Non-limiting examples of water-soluble solvents include, for example, glycerin, C₁₋₄ alcohols, organic solvents, polyols, glycols, and any a mixture thereof. Particularly useful water-soluble solvents include glycerin, mono-alcohols, polyols (polyhydric alcohols), glycols, and a mixture thereof. A more exhaustive but nonlimiting list of useful water-soluble solvents is provided below, under the heading “Water-Soluble Solvents.”

The amount of water-soluble solvents may vary, but typically the hair treatment compositions include about 0.1 to about 25 wt. %, of the one or more water-soluble solvents. Similarly, in some instances, the hair treatment compositions include about 0.1 to about 20 wt. %, about 0.1 to about 15 wt. %, about 0.1 to about 10 wt. %, about 0.1 to about 5 wt. %, about 0.5 to about 20 wt. %, about 0.5 to about 15 wt. %, about 0.5 to about 10 wt. %, about 0.5 wt. % to about 5 wt. %, including ranges and subranges therebetween, based on the total weight of the hair treatment composition. In some cases, higher amounts of water-soluble solvents may be used, for example, about 1 to about 25 wt. %, about 1 to about 20 wt. %, about 1 to about 15 wt. %, about 5 to about 25 wt. %, or about 5 to about 20 wt. %, including ranges and subranges therebetween, based on the total weight of the hair treatment composition.

The hair treatment compositions may optionally include one or more cationic conditioning polymers. Nonlimiting examples of cationic conditioning polymers include cationic cellulose derivatives, quaternized hydroxyethyl cellulose, cationic starch derivatives, cationic guar gum derivatives (e.g., hydroxypropyl guar hydroxypropyltrimonium chloride), cationic proteins and cationic protein hydrolysates (e.g., hydroxypropyl-trimonium hydrolyzed wheat protein), quaternary diammonium polymers (e.g., hexadimethrine chloride), copolymers of acrylamide and dimethyldiallyammonium chloride, polyquaterniums, and a mixture thereof. Particularly useful cationic conditioning polymers include guar gum derivatives, cationic proteins and cationic protein hydrolysates, and quaternary diammonium polymers, for example, hydroxypropyl guar hydroxypropyltrimonium chloride, hydroxypropyl-trimonium hydrolyzed wheat protein, and/or hexadimethrine chloride. A more exhaustive but nonlimiting list of useful cationic conditioning polymers is provided below, under the heading “Cationic Conditioning Polymers.”

The total amount of cationic conditioning polymers that may be included in the hair treatment compositions can vary, but typically, the hair treatment composition includes about 0.01 to about 10 wt. %, based on the total weight of the hair treatment composition. In some instances, the total amount of cationic conditioning polymers is about 0.01 to about 5 wt. %, about 0.01 to about 3 wt. %, about 0.01 to about 2 wt. %, about 0.01 to about 1 wt. %, about 0.1 to about 5 wt. %, about 0.1 to about 3 wt. %, about 0.1 to about 2 wt. %, about 0.1 to about 1 wt. %, including ranges and subranges therebetween, based on the total weight of the hair treatment composition.

The hair treatment compositions may optionally include one or more fatty compounds. Nonlimiting examples of fatty compounds include fatty alcohols, fatty acids, fatty esters, oils, waxes, alkanes (paraffins), triglyceride compounds, lanolin, derivatives thereof, and mixtures thereof. In some instances, fatty acids and/or fatty alcohols are particularly useful, for example, cetearyl alcohol. A more exhaustive but nonlimiting list of useful fatty compounds is provided below, under the heading “Fatty Compounds.”

The total amount of fatty compounds in the hair treatment compositions if present may vary. In some instances, the hair treatment compositions include about 0.1 to about 40 wt. % of one or more fatty compounds, based on the total weight of the hair treatment composition. Further, the hair treatment compositions may include about 0.1 to about 30 wt. %, about 0.1 to about 20 wt. %, about 0.1 to about 10 wt. %, about 0.1 to about 5 wt. %, about 0.1 to about 3 wt. %, about 1 to about 30 wt. %, about 1 to about 20 wt. %, about 1 to about 10 wt. %, about 1 to about 5 wt. %, about 1 to about 3 wt. %, about 2 to about 20 wt. %, about 2 to about 10 wt. %, or about 2 to about 5 wt. %, including ranges and subranges therebetween, based on the total weight of the hair treatment composition.

The hair treatment compositions may optionally include one or more silicones. For purposes of the instant disclosure silicones are distinct from fatty compounds, although silicones may share properties with fatty compounds, e.g., lack of solubility in water, etc. Non-limiting examples of silicones include dimethicone, cyclomethicone, polysilicone-11, phenyl trimethicone, trimethylsilylamodimethicone, and stearoxytrimethylsilane. In some instances, the one or more silicones are non-volatile silicon oils. In some embodiments, the silicone is polydimethylsiloxanes (PDMSs), polydimethylsiloxanes comprising alkyl or alkoxy groups which are pendent and/or at the end of the silicone chain, which groups each contain from 2 to 24 carbon atoms, or phenyl silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates. Other examples of silicone that may be mentioned include volatile linear or cyclic silicones, such as those with a viscosity 8 centistokes (8×106 m2/s) and/or containing from 2 to 7 silicon atoms. These silicones optionally comprise alkyl or alkoxy groups containing from 1 to 10 carbon atoms. Non-limiting examples of volatile silicone oils include octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, or mixtures thereof.

In some instances, the hair treatment compositions include one or more amino functionalized silicone. Nonlimiting examples include amodimethicone, bis-hydroxy/methoxy amodimethicones, bis-cetearyl amodimethicone, amodimethicone, bis(C13-15 alkoxy) PG amodimethicones, am inopropyl phenyl trimethicones, am inopropyl dimethicones, bis-amino PEG/PPG-41/3 am inoethyl PG-propyl dimethicones, caprylyl methicones, and a mixture thereof. Amodimethicone is a particularly useful amino functionalized silicone.

The total amount of silicones in the hair treatment composition may vary. In some instances, the amount of silicones in the hair treatment composition is about 0.01 to about 15 wt. %, about 0.01 to about 10 wt. %, about 0.01 to about 5 wt. %, about 0.01 to about 3 wt. %, about 0.01 to about 2 wt. %, about 0.1 to about 15 wt. %, about 0.1 to about 10 wt. %, about 0.1 to about 5 wt. %, about 0.1 to about 3 wt. %, about 0.1 to about 2 wt. %, about 1 to about 15 wt. %, about 1 to about 10 wt. %, about 1 to about 5 wt. %, about 1 to about 3 wt. %, or about 1 to about 2 wt. %, including ranges and subranges therebetween, based on the total weight of the hair treatment composition.

The hair treatment compositions may optionally include one or more miscellaneous ingredients. Nonlimiting examples include pH adjusters, fragrances, perfumes, colorants (to color the composition, not the hair), fruit and/or vegetable extracts, thickening agents, emulsifiers (that are different from the surfactants of the hair treatment compositions) proteins, polymers, preservatives, salts, emollients, fillers, acids (e.g., salicylic acid, citric acid, fumaric acid, sorbic acid, etc.), clays, hydrotropes, active ingredients (e.g., anti-dandruff agents, hair growth stimulating agents, anti-inflammatory agents, caffeine, panthenol, etc.), vitamins, etc.

The total amount of miscellaneous ingredient may vary but, in some instances, is about 0.001 to about 15 wt. %, based on the total weight of the hair treatment composition. The total amount of miscellaneous ingredients may be about 0.01 to about 15 wt. %, about 0.01 to about 10 wt. %, about 0.01 to about 8 wt. %, about 0.01 to about 5 wt. %, about 0.01 to about 3 wt. %, about 0.01 to about 2 wt. %, about 0.1 to about 15 wt. %, about 0.01 to about 10 wt. %, about 0.1 to about 10 wt. %, about 0.1 to about 8 wt. %, about 0.1 to about 5 wt. %, about 0.1 to about 3 wt. %, about 0.1 to about 2 wt. %, about 1 to about 15 wt. %, about 1 to about 10 wt. %, about 1 to about 8 wt. %, about 1 to about 5 wt. %, or about 1 to about 3 wt. %, including ranges and subranges therebetween, based on the total weight of the hair treatment composition.

The pH of the hair treatment compositions may vary, provided that the pH is safe for cosmetic purposes, i.e, for application to the hair, scalp, and skin. In some instances, the pH of the hair treatment composition is less than 7, for example, from about 3 to about 6.5, from about 3 to about 6, about 3 to about 5.5, about 3.5 to about 6.5, about 3.5 to about 6, about 3.5 to about 5.5, including ranges and subranges therebetween.

Nonlimiting examples of pH adjusters include organic acids, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and mixtures thereof.

In some instances, the hair treatment compositions may be in the form of an emulsion or in the form of a dispersion. In some instances, the hair treatment compositions are in the form of an emulsion such as an oil-in-water emulsion or a water-in-oil emulsion. In other instances, the hair treatment compositions are in the form of dispersions. In other instances, the hair treatment compositions are in the form of a lotion, gel, cream, milk, or rinse. Shampoos of the instant disclosure are often in the form of a dispersion. Conditioners of the instant disclosure, however, are often in the form of an emulsion.

All ingredients positively set forth throughout the instant disclosure may be negatively excluded from the hair treatment compositions. Nonetheless, in some instances, the hair treatment compositions are free from hair coloring dyes, hair coloring pigments, and hair coloring colorants. Hair coloring dyes, hair coloring pigments, and hair coloring colorants are ingredients used in compositions to purposely change the color of hair. Hair coloring dyes, hair coloring pigments, and hair coloring colorants are different from and distinguishable from dyes, pigments, and colorants that may be used in hair treatment compositions to impart a color or hue to the composition, but are not used to change or modify the color of the hair. Thus, hair treatment compositions that are free or essentially free from hair coloring dyes, hair coloring pigments, and hair coloring colorants may still include dyes, color, and pigments for modifying the color or hue of the hair treatment composition provided that they do not change the color of the hair (they merely change the color of the composition). Accordingly, in some instances, the hair treatment compositions are not hair coloring or hair bleaching compositions. Furthermore, the hair treatment compositions may be free or essentially free from oxidizing agents, such as peroxides. Similarly, in some instances, the hair treatment compositions are not permanent hair shaping compositions, for example, hair straightening and/or hair curling compositions.

In certain instances, the hair treatment compositions comprise or consists of:
(a) about 0.1 to about 5 wt. %, preferably about 0.1 to about 3 wt. %, more preferably about 0.1 to about 2 wt. %, of the ascorbyl glucoside;
(b) about 0.01 to about 3 wt. %, preferably about 0.05 to about 2 wt. %, more preferably about 0.1 to about 1 wt. %, of the amino acid chelant, for example, glycine, lysine, methionine, and combinations thereof, preferably glycine;
(c) about 0.01 to about 3 wt. %, preferably about 0.05 to about 2 wt. %, more preferably about 0.1 to about 1 wt. %, of N,N-dicarboxymethyl glutamic acid, a salt thereof, and/or a combination thereof, preferably tetrasodium glutamate diacetate;
(d) about 0.5 to about 25 wt. %, preferably about 0.5 to about 20 wt. %, more preferably about 1 to about 15 wt. %, of the one or more surfactants selected from anionic, cationic, nonionic, amphoteric, and combinations thereof; and
(e) about 50 to about 95 wt. %, preferably about 65 to about 95 wt. %, more preferably about 75 to 92 wt. %, of the water;
(f) optionally, about 0.1 to about 25 wt. %, preferably about 0.1 to about 10 wt. %, more preferably, about 1 to about 5 wt. % of one or more water-soluble solvents, for example, chosen from glycerin, mono-alcohols, polyols (polyhydric alcohols), glycols, and a mixture thereof, preferably, chosen from glycerin, monoalcohol, polyhydric alcohol, glycols, and mixtures thereof, more preferably, chosen from glycerin, isopropyl alcohol, hexylene glycol, and mixtures thereof;
(g) optionally, about 0.01 to about 10, preferably, about 0.01 to about 5, more preferably about 0.1 to about 3 wt. % of one or more cationic conditioning polymers, for example, one or more cationic conditioning polymers chosen from cationic cellulose derivatives, quaternized hydroxyethyl cellulose, cationic starch derivatives, cationic guar gum derivatives (e.g., hydroxypropyl guar hydroxypropyltrimonium chloride), cationic proteins and cationic protein hydrolysates (e.g., hydroxypropyltrimonium hydrolyzed wheat protein), quaternary diammonium polymers (e.g., hexadimethrine chloride), copolymers of acrylamide and dimethyldiallyammonium chloride, polyquaterniums, and a mixture thereof, preferably hydroxypropyl guar hydroxypropyltrimonium chloride;
(h) optionally, about 0.01 to about 25 wt. %, preferably about 0.1 to about 15 wt. %, more preferably, about 1 to about 10 wt. % of one or more fatty compounds, wherein at least one of the fatty compounds is optionally a fatty alcohol, preferably, cetearyl alcohol; and
(i) optionally, about 0.01 to about 20 wt. %, preferably about 0.1 to about 15 wt. %, more preferably about 0.1 to about 5 wt. %, of one or more silicones, preferably and amino functionalized silicone such as amodimethicone;
(j) optionally, about 0.1 to about 15 wt. %, preferably about 0.1 to about 10 wt. %, more preferably about 0.1 to about 5 wt. % of one or more miscellaneous ingredients;
   wherein all weight percentages are based on the total weight of the hair treatment composition.

The pH and/or the viscosity of the hair treatment compositions may be as set forth above. Similarly, as set forth above, the hair treatment compositions may be free or essentially free from coloring dyes, hair coloring pigments, and hair coloring colorants, and/or free or essentially free of ascorbic acid (vitamin C).

Shampoos according to the disclosure may comprise or consist of:
(a) about 0.1 to about 5 wt. %, preferably about 0.1 to about 3 wt. %, more preferably about 0.1 to about 2 wt. %, of the ascorbyl glucoside;
(b) about 0.01 to about 3 wt. %, preferably about 0.05 to about 2 wt. %, more preferably about 0.1 to about 1 wt. %, of the amino acid chelant, for example, glycine, lysine, methionine, and combinations thereof, preferably glycine;
(c) about 0.01 to about 3 wt. %, preferably about 0.05 to about 2 wt. %, more preferably about 0.1 to about 1 wt. %, of N,N-dicarboxymethyl glutamic acid, a salt thereof, and/or a combination thereof, preferably tetrasodium glutamate diacetate;
(d) about 0.5 to about 25 wt. %, preferably about 0.5 to about 20 wt. %, more preferably about 1 to about 20 wt. %, of the one or more anionic surfactants selected from alkyl sulfates, alkyl ether sulfates, alkyl sulfonates, alkyl sulfosuccinates, alkyl sulfoacetates, acyl isethionates, alkoxylated monoacids, acyl amino acids such as acyl taurates, acyl glycinates, acyl glutamates, acyl sarcosinates, salts thereof, and a mixture thereof, preferably one more alkyl sulfates such as sodium lauryl sulfate (SLS) and/or sodium dodecyl sulfate (SDS); and
   optionally, about 0.1 to about 15 wt. %, preferably about 0.1 to about 10 wt. %, more preferably about 1 to about 5 wt. % of one or more amphoteric surfactants, preferably one or more betaines; and
   optionally, about 0.1 to about 15 wt. %, preferably about 0.1 to about 10 wt. %, more preferably about 1 to about 5 wt. % of one or more nonionic surfactants;
(e) about 50 to about 95 wt. %, preferably about 60 to about 92 wt. %, more preferably about 70 to 85 wt. %, of water;
(f) optionally, about 0.1 to about 25 wt. %, preferably about 0.1 to about 10 wt. %, more preferably, about 1 to about 5 wt. % of one or more water-soluble solvents, for example, chosen from glycerin, mono-alcohols, polyols (polyhydric alcohols), glycols, and a mixture thereof, preferably, chosen from glycerin, monoalcohol, polyhydric alcohol, glycols, and mixtures thereof, more preferably, chosen from glycerin, isopropyl alcohol, hexylene glycol, and mixtures thereof;
(g) optionally, about 0.01 to about 10, preferably, about 0.01 to about 5, more preferably about 0.1 to about 3 wt. % of one or more cationic conditioning polymers, for example, one or more cationic conditioning polymers chosen from cationic cellulose derivatives, quaternized hydroxyethyl cellulose, cationic starch derivatives, cationic guar gum derivatives (e.g., hydroxypropyl guar hydroxypropyltrimonium chloride), cationic proteins and cationic protein hydrolysates (e.g., hydroxypropyltrimonium hydrolyzed wheat protein), quaternary diammonium polymers (e.g., hexadimethrine chloride), copolymers of acrylamide and dimethyldiallyammonium chloride, polyquaterniums, and a mixture thereof, preferably hydroxypropyl guar hydroxypropyltrimonium chloride;
(h) optionally, about 0.01 to about 25 wt. %, preferably about 0.1 to about 15 wt. %, more preferably, about 1 to about 10 wt. % of one or more fatty compounds, wherein at least one of the fatty compounds is optionally a fatty alcohol, preferably, cetearyl alcohol; and
(i) optionally, about 0.01 to about 20 wt. %, preferably about 0.1 to about 15 wt. %, more preferably about 0.1 to about 5 wt. %, of one or more silicones, preferably and amino functionalized silicone such as amodimethicone;
(j) optionally, about 0.1 to about 15 wt. %, preferably about 0.1 to about 10 wt. %, more preferably about 0.1 to about 5 wt. % of one or more miscellaneous ingredients;
   wherein all weight percentages are based on the total weight of the shampoo.

The pH of the shampoo is typically less than 7, for example, from about 3 to about 6.5, from about 3 to about 6, about 3 to about 5.5, about 3.5 to about 6.5, about 3.5 to about 6, about 3.5 to about 5.5, including ranges and subranges therebetween. The viscosity of the shampoo can be from about 2500 cP to about 15,000 cp at a temperature of 25° C. In addition, the shampoo may be free or essentially free from coloring dyes, hair coloring pigments, and/or hair coloring colorants. It is preferred that the shampoo is in the form of a dispersion.

Conditioners according to the disclosure may comprises or consist of:
(a) about 0.1 to about 5 wt. %, preferably about 0.1 to about 3 wt. %, more preferably about 0.1 to about 2 wt. %, of the ascorbyl glucoside;
(b) about 0.01 to about 3 wt. %, preferably about 0.05 to about 2 wt. %, more preferably about 0.1 to about 1 wt. %, of the amino acid chelant, for example, glycine, lysine, methionine, and combinations thereof, preferably glycine;
(c) about 0.01 to about 3 wt. %, preferably about 0.05 to about 2 wt. %, more preferably about 0.1 to about 1 wt. %, of N,N-dicarboxymethyl glutamic acid, a salt thereof, and/or a combination thereof, preferably tetrasodium glutamate diacetate;
(d) about 0.1 to about 20 wt. %, preferably about 0.1 to about 10 wt. %, more preferably about 1 to about 5 wt. %, of one or more cationic surfactants, for example, cationic surfactants chosen from cetrimonium chloride, stearimonium chloride, behentrimonium chloride, behentrimonium methosulfate, behenam idopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, and mixtures thereof, preferably one or more cationic surfactants chosen from cetrimonium chloride, behentrimonium chloride, behentrimonium methosulfate, and combinations thereof;
   optionally, about 0.1 to about 15 wt. %, preferably about 0.1 to about 10 wt. %, more preferably about 1 to about 5 wt. % of one or more amphoteric surfactants; and
   optionally, about 0.1 to about 15 wt. %, preferably about 0.1 to about 10 wt. %, more preferably about 1 to about 5 wt. % of one or more nonionic surfactants;
(e) about 50 to about 95 wt. %, preferably about 70 to about 95 wt. %, more preferably about 80 to 92 wt. % of water;
(f) optionally, about 0.1 to about 25 wt. %, preferably about 0.1 to about 10 wt. %, more preferably, about 1 to about 5 wt. % of one or more water-soluble solvents, for example, chosen from glycerin, mono-alcohols, polyols (polyhydric alcohols), glycols, and a mixture thereof, preferably, chosen from glycerin, monoalcohol, polyhydric alcohol, glycols, and mixtures thereof, more preferably, chosen from glycerin, isopropyl alcohol, hexylene glycol, and mixtures thereof;
(g) optionally, about 0.01 to about 10, preferably, about 0.01 to about 5, more preferably about 0.1 to about 3 wt. % of one or more cationic conditioning polymers, for example, one or more cationic conditioning polymers chosen from cationic cellulose derivatives, quaternized hydroxyethyl cellulose, cationic starch derivatives, cationic guar gum derivatives (e.g., hydroxypropyl guar hydroxypropyltrimonium chloride), cationic proteins and cationic protein hydrolysates (e.g., hydroxypropyltrimonium hydrolyzed wheat protein), quaternary diammonium polymers (e.g., hexadimethrine chloride), copolymers of acrylamide and dimethyldiallyammonium chloride, polyquaterniums, and a mixture thereof, preferably hydroxypropyl guar hydroxypropyltrimonium chloride;
(h) optionally, about 0.01 to about 25 wt. %, preferably about 0.1 to about 15 wt. %, more preferably, about 1 to about 10 wt. % of one or more fatty compounds, wherein at least one of the fatty compounds is optionally a fatty alcohol, preferably, cetearyl alcohol; and
(i) optionally, about 0.01 to about 20 wt. %, preferably about 0.1 to about 15 wt. %, more preferably about 0.1 to about 5 wt. %, of one or more silicones, preferably and amino functionalized silicone such as amodimethicone;
(j) optionally, about 0.1 to about 15 wt. %, preferably about 0.1 to about 10 wt. %, more preferably about 0.1 to about 5 wt. % of one or more miscellaneous ingredients;
   wherein all weight percentages are based on the total weight of the conditioner.

The pH of the conditioner is typically less than 7, for example, from about 3 to about 6.5, from about 3 to about 6, about 3 to about 5.5, about 3.5 to about 6.5, about 3.5 to about 6, about 3.5 to about 5.5, including ranges and subranges therebetween. The viscosity of the conditioner can be from about 2500 cP to about 15,000 cp at a temperature of 25° C. In addition, the shampoo may be free or essentially free from coloring dyes, hair coloring pigments, and hair coloring colorants, and/or free or essentially free of ascorbic acid (vitamin C). It is preferred that the conditioner is in the form of an emulsion.

The hair treatment compositions of the instant disclosure may be provided in a kit, for example, a kit comprising an individually contained hair treatment composition according to the instant disclosure and one or more additional separately contained compositions. The one or more separately contained compositions may be another composition according to the instant disclosure or may be a different composition. The one or more additional separately contained compositions may be, for example, a shampoo, a conditioner, a hair styling composition, a hair bleaching (lightening) composition and/or a hair coloring composition (for example containing an oxidizing agent and/or a oxidative dye and/or an oxidative dye precursor), a hair shaping composition (for example, a relaxer, straightener, and/or curling composition), etc.

In some instances, the kit may include a shampoo according to the instant disclosure, and one or more separately contained compositions, for example a conditioner. The separately contained conditioner may be a conditioner according to the instant disclosure or may be an independent conditioner. Similarly, the kit may include a conditioner according to the instant disclosure, and one or more separately contained compositions, for example a shampoo. The separately contained shampoo may be a shampoo according to the instant disclosure or may be an independent shampoo. In some instances, the kit includes both a shampoo according to the instant disclosure and a conditioner according to the instant disclosure, wherein the shampoo and the conditioner are separately contained.

The instant disclosure also relates to methods of treating hair using the hair treatment compositions according to the instant disclosure. The methods include applying a hair treatment composition according to the disclosure to the hair, optionally allowing the hair treatment composition to remain on the hair for a period, and subsequently rinsing the hair treatment composition from the hair. The hair treatment compositions are typically applied to wet or damp hair but may be applied to dry hair. The hair treatment compositions are typically more effective if massaged or lathered into the hair before rinsing. Typically, the hair treatment compositions are allowed to remain on the hair in the same manner a typical shampoo and/or conditioner are allowed to remain on the hair. For example, the hair treatment compositions may be allowed to remain on the hair for about 5 seconds to about 1 hour, about 5 seconds to about 30 minutes, about 5 seconds to about 15 minutes, about 5 seconds to about 10 minutes, about 5 seconds to about 5 minutes, including all ranges and subranges therebetween. The process of treating the hair (i.e., applying the hair treatment composition to the hair, optionally allowing the hair treatment composition to remain on the hair for a period, and rinsing the hair treatment composition from the hair) can be carried out once, or may be carried out multiple times. For example, the process of treating the hair may be carried out daily, weekly, monthly for an extended period of time, for example, for a week (daily for a week), for 1, 2, 3, 4, 5, or 6 months (daily or weekly for a number of months), or monthly for a period of 1, 2, 3, 4, 5, of 6 months (about once or twice a month for a number of months).

An important method according to the disclosure includes treating the hair with a shampoo according to the instant disclosure and with a conditioner according to the instant disclosure. These shampoo and conditioners are typically used according to a consumers regular shampooing and conditioning habits, e.g., daily, 2, 3, 4, 5, or 6 times per week. Using both a shampoo and a conditioner according to the instant disclosure together can enhance the brightening, shine, luster, and cleanliness and/or condition provided to hair provided to the hair. Thus, the methods according to the disclosure encompass methods for brightening the hair, methods for enhancing the shine of the hair, methods for improving the luster of the hair, and/or methods for cleaning and/or conditioning the hair.

Water-Soluble Solvents

Non-limiting examples of water-soluble solvents include, for example, alcohols (for example, C₁₋₁₅, C₁₋₁₀, or C₁₋₆ alcohols), polyols (polyhydric alcohols and glycols (e.g., glycerin, butylene glycol, caprylyl glycol, etc.), and a mixture thereof. The water-soluble-solvents may be referred to as organic solvents. Non-limiting examples include monoalcohols and polyols such as ethyl alcohol, isopropyl alcohol, propyl alcohol, benzyl alcohol, and phenylethyl alcohol, or glycerin or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of water-soluble solvents are ethylene glycol, propylene glycol, butylene glycol, hexylene glycol, propane diol, and glycerin. The water-soluble solvents can be volatile or non-volatile compounds.

Further non-limiting examples include alkanediols such as 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

Polyhydric alcohols are useful. Examples of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof.

Anionic Surfactants

Alkyl Sulfates and Alkyl Ether Sulfates

Useful alkyl sulfates include C₈₋₁₈alky sulfates, more preferably C₁₂₋₁₈ alkyl sulfates, preferably in the form of a salt with a solubilizing cation such as sodium, potassium, ammonium or substituted ammonium. Examples are sodium lauryl sulfate (SLS) or sodium dodecyl sulfate (SDS). Useful alkyl ether sulfates include those having the formula: RO(CH₂CH₂O)ₙSO₃M; wherein R is an alkyl or alkenyl having from 8 to 18 (preferably 12 to 18) carbon atoms; n is a number having an average value of greater than at least 0.5, preferably between 1 and 3, more preferably between 2 and 3; and M is a solubilizing cation such as sodium, potassium, ammonium or substituted ammonium. An example is sodium lauryl ether sulfate (SLES).

In some instances, useful alkyl sulfate salts and alkyl ether sulfate salts include those having the formulas (I and II):

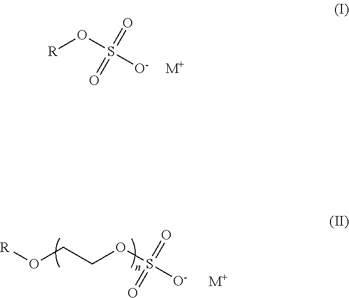

wherein, R is alkyl chain having 6 to 24 carbon atoms, preferably 8 to 18 carbon atoms, and more preferably 12 to 18 carbon atoms;

M is a solubilizing cation such as alkali metal ions such as sodium or potassium, ammonium ions, or alkanolammonium ions such as monoethanolammonium or triethanolammonium ions; and

n is an integer from 0 to 3.

Alkyl Sulfonates

Useful alkyl sulfonates include alkyl aryl sulfonates, primary alkane disulfonates, alkene sulfonates, hydroxyalkane sulfonates, alkyl glyceryl ether sulfonates, alpha-olefinsulfonates, sulfonates of alkylphenolpolyglycol ethers, alkylbenzenesulfonates, phenvlalkanesulfonates, alpha-olefinsulfonates, olefin sulfonates, alkene sulfonates, hydroxyalkanesulfonates and disulfonates, secondary alkanesulfonates, paraffin sulfonates, ester sulfonates, sulfonated fatty acid glycerol esters, and alpha-sulfo fatty acid methyl esters including methyl ester sulfonate.

In some instances, an alkyl sulfonate of formula (III) is particularly useful.

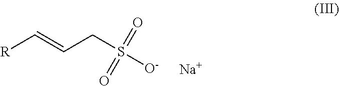

R is selected from H or alkyl chain that has 1-24 carbon atoms, preferably 6-24 carbon atoms, more preferably, 8 to 20 carbon atoms, said chain being saturated or unsaturated, linear or branched. Sodium is shown as the cation in the above formula (III) but the cation may be an alkali metal ion such as sodium or potassium, ammonium ions, or alkanolammonium ions such as monoethanolammonium or triethanolammonium ions. In some instances, the alkyl sulfonate(s) are selected from C₈-C₁₆ alkyl benzene sulfonates, C₁₀-C₂₀ paraffin sulfonates, C₁₀-C₂₄ olefin sulfonates, salts thereof, and mixtures thereof. C₁₀-C₂₄ olefin sulfonates are particularly preferred.

A non-limiting but particularly useful example of a C₁₀-C₂₄ olefin sulfonate that can be used in the instant compositions is sodium C14-16 olefin sulfonate.

Alkyl Sulfosuccinates

Non-limiting examples of useful sulfosuccinates include those of formula (IV):

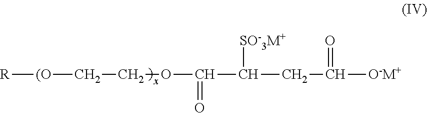

wherein R is a straight or branched chain alkyl or alkenyl group having 10 to 22 carbon atoms, preferably 10 to 20 carbon atoms, X is a number that represents the average degree of ethoxylation and can range from 0 to about 5, preferably from 0 to about 4, and most preferably from about 2 to about 3.5, and M and M′ are monovalent cations which can be the same or different from each other. Preferred cations are alkali metal ions such as sodium or potassium, ammonium ions, or alkanolammonium ions such as monoethanolammonium or triethanolammonium ions.

Non-limiting examples of alkyl sulfosuccinates salts include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, and a mixture thereof. In some instances, disodium laureth sulfosuccinate is particularly preferred.

Alkyl Sulfoacetates

Non-limiting examples of alkyl sulfacetates includes, for example, alkyl sulfoacetates such as C4-C18 fatty alcohol sulfoacetates and/or salts thereof. A particularly preferred sulfoacetate salt is sodium lauryl sulfoacetate. Useful cations for the salts include alkali metal ions such as sodium or potassium, ammonium ions, or alkanolammonium ions such as monoethanolammonium or triethanolammonium ions.

Acyl Isethionates

Non-limiting examples of useful acyl isethionates include those of formula (V) and (VI):

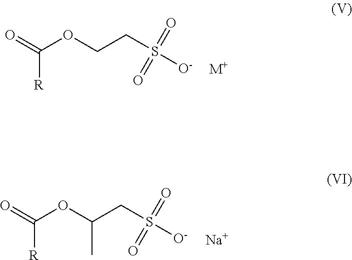

wherein R, R¹, R² and R³ are each independently selected from H or an alkyl chain having 1-24 carbon atoms, said chain being saturated or unsaturated, linear or branched, and X is COO⁻ or SO₃⁻. Sodium is shown as the cation in formula (VI) but the cation for both formula (V) and formula (VI) may be an alkali metal ion such as sodium or potassium, ammonium ions, or alkanolammonium ions such as monoethanolammonium or triethanolammonium ions. Non-limiting examples of acyl isethionates include sodium isethionate, sodium cocoyl isethionate, sodium lauroyl methyl isethionate, and sodium cocoyl methyl isethionate.

Alkoxylated Monoacids

Non-limiting examples of alkoxylated monoacids include compounds corresponding to formula (VII):

RO[CH2O]u[(CH2)xCH(R′)(CH2)y(CH2)zO]v[CH2CH2O]wCH2COOH (VII)

wherein:

R is a hydrocarbon radical containing from about 6 to about 40 carbon atoms;

u, v and w, independently of one another, represent numbers of from 0 to 60;

x, y and z, independently of one another, represent numbers of from 0 to 13;

R′ represents hydrogen, alkyl, and

the sum of x+y+z>0;

Compounds corresponding to formula (VII) can be obtained by alkoxylation of alcohols ROH with ethylene oxide as the sole alkoxide or with several alkoxides and subsequent oxidation. The numbers u, v, and w each represent the degree of alkoxylation. Whereas, on a molecular level, the numbers u, v and w and the total degree of alkoxylation can only be integers, including zero, on a macroscopic level they are mean values in the form of broken numbers.

In formula (VII), R is linear or branched, acyclic or cyclic, saturated or unsaturated, aliphatic or aromatic, substituted or unsubstituted. Typically, R is a linear or branched, acyclic C6-40 alkyl or alkenyl group or a C1-40 alkyl phenyl group, more typically a C8-22 alkyl or alkenyl group or a C4-18 alkyl phenyl group, and even more typically a C12-18 alkyl group or alkenyl group or a C6-16 alkyl phenyl group; u, v, w, independently of one another, is typically a number from 2 to 20, more typically a number from 3 to 17 and most typically a number from 5 to 15; x, y, z, independently of one another, is typically a number from 2 to 13, more typically a number from 1 to 10 and most typically a number from 0 to 8.

Suitable alkoxylated monoacids include, but are not limited to: Butoxynol-5 Carboxylic Acid, Butoxynol-19 Carboxylic Acid, Capryleth-4 Carboxylic Acid, Capryleth-6 Carboxylic Acid, Capryleth-9 Carboxylic Acid, Ceteareth-25 Carboxylic Acid, Coceth-7 Carboxylic Acid, C9-11 Pareth-6 Carboxylic Acid, C11-15 Pareth-7 Carboxylic Acid, C12-13 Pareth-5 Carboxylic Acid, C12-13 Pareth-8 Carboxylic Acid, C12-13 Pareth-12 Carboxylic Acid, C12-15 Pareth-7 Carboxylic Acid, C12-15 Pareth-8 Carboxylic Acid, C14-15 Pareth-8 Carboxylic Acid, Deceth-7 Carboxylic Acid, Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, PPG-6-Laureth-6 Carboxylic Acid, PPG-8-Steareth-7 Carboxylic Acid, Myreth-3 Carboxylic Acid, Myreth-5 Carboxylic Acid, Nonoxynol-5 Carboxylic Acid, Nonoxynol-8 Carboxylic Acid, Nonoxynol-10 Carboxylic Acid, Octeth-3 Carboxylic Acid, Octoxynol-20 Carboxylic Acid, Oleth-3 Carboxylic Acid, Oleth-6 Carboxylic Acid, Oleth-10 Carboxylic Acid, PPG-3-Deceth-2 Carboxylic Acid, Capryleth-2 Carboxylic Acid, Ceteth-13 Carboxylic Acid, Deceth-2 Carboxylic Acid, Hexeth-4 Carboxylic Acid, Isosteareth-6 Carboxylic Acid, Isosteareth-11 Carboxylic Acid, Trudeceth-3 Carboxylic Acid, Trideceth-6 Carboxylic Acid, Trideceth-8 Carboxylic Acid, Trideceth-12 Carboxylic Acid, Trideceth-3 Carboxylic Acid, Trideceth-4 Carboxylic Acid, Trideceth-7 Carboxylic Acid, Trideceth-15 Carboxylic Acid, Trideceth-19 Carboxylic Acid, Undeceth-5 Carboxylic Acid and mixtures thereof. In some cases, preferred ethoxylated acids include Oleth-10 Carboxylic Acid, Laureth-5 Carboxylic Acid, Laureth-11 Carboxylic Acid, and a mixture thereof.

Acyl Amino Acids

Acyl amino acids that may be used include, but are not limited to, amino acid surfactants based on alanine, arginine, aspartic acid, glutamic acid, glycine, isoleucine, leucine, lysine, phenylalanine, serine, tyrosine, valine, sarcosine, threonine, and taurine. The most common cation associated with the acyl amino acid can be sodium or potassium. Alternatively, the cation can be an organic salt such as triethanolamine (TEA) or a metal salt. Non-limiting examples of useful acyl amino acids include those of formula (VIII):

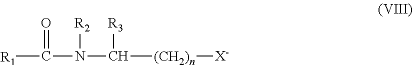

wherein R, R¹, R² and R³ are each independently selected from H or an alkyl chain having 1-24 carbon atoms, said chain being saturated or unsaturated, linear or branched, and X is COO⁻ or S₃⁻.

Acyl Taurates: Non-limiting examples of acyl taurates include those of formula (IX):

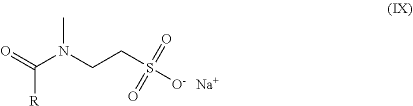

wherein R, R¹, R² and R³ are each independently selected from H or an alkyl chain having 1-24 carbon atoms, or from 6-20 carbon atoms, or from 8 to 16 carbon atoms, said chain being saturated or unsaturated, linear or branched, and X is COO⁻ or SO₃⁻. Non-limiting examples of acyl taurate salts include sodium cocoyl taurate and sodium methyl cocoyl taurate.

Acyl Glycinates: Non-limiting examples of useful acyl glycinates include those of formula (X):

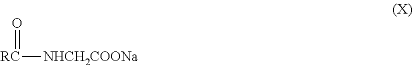

wherein R is an alkyl chain of 8 to 16 carbon atoms. Sodium is shown as the cation in the above formula (X) but the cation may be an alkali metal ion such as sodium or potassium, ammonium ions, or alkanolammonium ions such as monoethanolammonium or triethanolammonium ions. Non-limiting examples of acyl glycinates include sodium cocoyl glycinate, sodium lauroyl glycinate, sodium myristoyl glycinate, potassium lauroyl glycinate, and potassium cocoyl glycinate, and in particular sodium cocoyl glycinate.

Acyl Glutamates: Non-limiting examples of useful acyl glutamates include those of formula (XI):

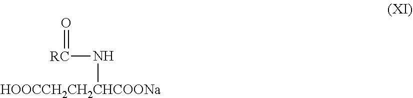

wherein R is an alkyl chain of 8 to 16 carbon atoms. Sodium is shown as the cation in the above formula (XI) but the cation may be an alkali metal ion such as sodium or potassium, ammonium ions, or alkanolammonium ions such as monoethanolammonium or triethanolammonium ions. Non-limiting examples of acyl gluatamtes include dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, triethanolamine mono-cocoyl glutamate, triethanolamine lauroylglutamate, and disodium cocoyl glutamate. In some cases, sodium stearoyl glutamate is particularly preferred.

Acyl Sarcosinates: Non-limiting examples of acyl sarcosinates include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, and ammonium lauroyl sarcosinate.

### Cationic Surfactants

Non-limiting examples of cationic surfactants include cetrimonium chloride, stearimonium chloride, behentrimonium chloride, behentrimonium methosulfate, behenam idopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidam ido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, and mixtures thereof.

In some embodiments, the cationic surfactant is selected from cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleam idopropyl dimethylamine, isostearam idopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, and mixtures thereof.

In some embodiments, the cationic surfactant comprises cetrimonium chloride, behentrimonium chloride, and mixtures thereof. Behentrimonium Chloride, also described by the technical names that include 1-Docosanaminium, N,N,N-Trimethyl-, Chloride, and N,N,N-Trimethyl-1-Docosanaminium Chloride, is the quaternary ammonium salt that conforms to the formula:

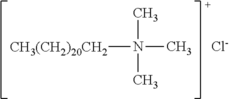

Additional nonlimiting examples of cationic surfactants include behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride(Quaternium-14), Quaternium-22, Quaternium-26, Quaternium-18 hectorite, dimethylaminoethylchloride hydrochloride, cysteine hydrochloride, diethanolammonium

POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

The cationic surfactant(s) may also be chosen from optionally polyoxyalkylenated, primary, secondary or tertiary fatty amines, or salts thereof, and quaternary ammonium salts, and mixtures thereof.

In some cases, it is useful to use salts such as chloride salts of the quaternary ammonium compounds.

The fatty amines generally comprise at least one C₈-C₃₀ hydrocarbon-based chain.

Examples of quaternary ammonium salts that may especially be mentioned include: those corresponding to the general formula below:

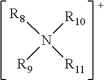

in which the groups R₈ to R₁₁, which may be identical or different, represent a linear or branched, saturated or unsaturated aliphatic group comprising from 1 to 30 carbon atoms, or an aromatic group such as aryl or alkylaryl, at least one of the groups R₈ to R₁₁ denoting a group comprising from 8 to 30 carbon atoms and, in some embodiments, from 12 to 24 carbon atoms. The aliphatic groups may comprise heteroatoms especially such as oxygen, nitrogen, sulfur and halogens. The aliphatic groups are chosen, for example, from C₁-C₃₀ alkyl, C₂-C₃₀ alkenyal, C₁-C₃₀ alkoxy, polyoxy(C₂-C₆)alkylene, alkylamide, (C₁₂-C₂₂)alkylamido(C₂-C₆)alkyl, (C₁₂-C₂₂)alkyl acetate and C₁-C₃₀ hydroxyalkyl groups; X⁻ is an anion chosen from the group of halides, phosphates, acetates, lactates, (C₁-C₄)alkyl sulfates, and (C₁-C₄)alkyl- or (C₁-C₄)alkylarylsulfonates.

Among the quaternary ammonium salts of formula (III), those that are preferred are, on the one hand, tetraalkylammonium salts, for instance dialkyldimethylammonium or alkyltrimethylammonium salts in which the alkyl group contains approximately from 12 to 22 carbon atoms, in particular behenyltrimethylammonium, distearyldimethylammonium, cetyltrimethylammonium or benzyldimethylstearylammonium salts, or, on the other hand, oleocetyldimethylhydroxyethylammonium salts, palmitylamidopropyltrimethylammonium salts, stearamidopropyltrimethylammonium salts and stearamidopropyldimethylcetearylammonium salts.

Also useful are quaternary ammonium salts of imidazoline, such as, for example, those of formula below:

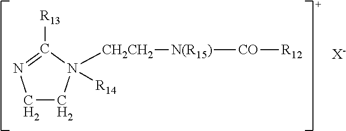

in which R₁₂ represents an alkenyl or alkyl group comprising from 8 to 30 carbon atoms, derived for example from tallow fatty acids, R₁₃ represents a hydrogen atom, a C₁-C₄ alkyl group or an alkyl or alkenyl group comprising from 8 to 30 carbon atoms, R₁₄ represents a C₁-C₄ alkyl group, R₁₅ represents a hydrogen atom or a C₁-C₄ alkyl group, X⁻ is an anion chosen from the group of halides, phosphates, acetates, lactates, alkyl sulfates, alkyl- or alkylaryl-sulfonates in which the alkyl and aryl groups, in some embodiments, comprise, respectively, from 1 to 20 carbon atoms and from 6 to 30 carbon atoms. R₁₂ and R₁₃, in some embodiments, denote a mixture of alkenyl or alkyl groups containing from 12 to 21 carbon atoms, derived for example from tallow fatty acids, R₁₄, in some embodiments, denotes a methyl group, and R₁₅, in some embodiments, denotes a hydrogen atom. Such a product is sold, for example, under the name REWOQUAT W 75 by the company Rewo.

Useful quaternary diammonium or triammonium salts includes those of the formula:

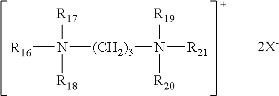

in which R₁₆ denotes an alkyl radical comprising approximately from 16 to 30 carbon atoms, which is optionally hydroxylated and/or interrupted with one or more oxygen atoms, R₁₇ is chosen from hydrogen or an alkyl radical comprising from 1 to 4 carbon atoms or a group (R₁₆ₐ)(R₁₇ₐ)(R₁₈ₐ)N—(CH₂)₃, R₁₆ₐ, R₁₇ₐ, R₁₈ₐ, R₁₈, R₁₉, R₂₀ and R₂₁, which may be identical or different, being chosen from hydrogen and an alkyl radical comprising from 1 to 4 carbon atoms, and X⁻ is an anion chosen from the group of halides, acetates, phosphates, nitrates and methyl sulfates. Such compounds are, for example, Finquat CT-P, sold by the company Finetex (Quaternium 89), and Finquat CT, sold by the company Finetex (Quaternium 75).

Useful cationic/cationizable surfactants, including cationizable surfactants together with an acid neutralizer, include those of the general structure R4-A-R5-B wherein R4 is a saturated or unsaturated, straight or branched alkyl chain with 8 to 24 C atoms, R5 is a straight or branched alkyl chain with 1 to 4 C atoms, A is selected from:

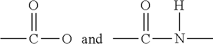

and B is selected from

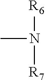

wherein R₆ and R₇ are the same or different is H or an alkyl chain with 1 to 4 C atoms, hydroxyl alkyl chain with 1 to 4 C atoms and di hydroxyl alkyl chain with 2 to 4 C atoms, and

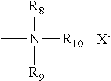

wherein R₈ and R₉ are the same or different, an alkyl chain with 1 to 4 C atoms, hydroxyl alkyl chain with 1 to 4 C atoms and di hydroxyl alkyl chain with 2 to 4 C atoms, R.sub.10 is an alkyl chain with 1 to 4 C atoms, hydroxyl alkyl chain with 1 to 4 C atoms or di hydroxyl alkyl chain with 2 to 4 C atoms.

In some instances, R₄ is saturated or unsaturated, straight or branched alkyl chain with 10 to 24C atoms, in some embodiments, 12 to 22 C atoms and R₅ is straight or branched alkyl group with 1 to 4 C atoms, and A, B, R₆ to R₁₀ are same as above.

Non-limiting suitable examples are stearyloxypropyl amine, palmityloxypropyl amine, stearyloxypropyldimethyl amine, stearyloxypropyldiethyl amine, stearyloxyethylyldimethyl amine, stearyloxyethyl amine, myristyloxypropyl amine, myristyloxypropyldimethyl amine, palmitamidopropyl amine, palmitamidopropyl methylamine, palmitamidopropyl diethylamine, palmitamidopropyl dibutylamine, palm itam idopropyl buylamine, palmitamidopropyl dipropylamine, palmitamidopropyl propylamine, palmitamidopropyl dihydroxyethylamine, palm itam idopropyl hydroxyethylamine, palmitamidopropyl dihydroxypropylamine, palmitamidopropyl hydroxypropylamine, lauramidopropyl amine, lauramidopropyl methylamine, lauramidopropyl diethylamine, lauramidopropyl dibutylamine, lauramidopropyl buylamine, lauramidopropyl dipropylamine, lauramidopropyl propylamine, lauramidopropyl dihydroxyethylamine, lauramidopropyl hydroxyethylamine, lauramidopropyl dihydroxypropylamine, lauramidopropyl hydroxypropylamine, stearamidopropyl amine, stearamidopropyl dimethylamine, steara midopropyl diethylamine, stearamidopropyldibutylamine, stearamidopropyl butylamine, stearamidopropyl dipropylamine, behenamidopropyl propylamine, behenamidopropyl dihydroxyethylamine, behenamidopropyl hydroxyethylamine, behenamidopropyl dihydroxypropylamine, behenamidopropyl hydroxypropylamine, behenamidopropyl amine, behenamidopropyl methylamine, behenamidopropyl diethylamine, behenamidopropyl dibutylamine, behenamidopropyl butylamine, behenamidopropyl dipropylamine, behenamidopropyl propylamine, behenamidopropyl dihydroxyethylamine, behenamidopropyl hydroxyethylamine, behenamidopropyl dihydroxypropylamine, behenamidopropyl hydroxypropylamine, dipalmitamidopropyl methylamine, dipalmitamidopropyl ethylamine, dipalmitamidopropyl butylamine, dipalmitamidopropyl propylamine, dipalmitamidopropyl hydroxyethylamine, dipalmitamidopropyl hydroxypropylamine, dilauramidopropyl amine, dilauramidopropyl methylamine, dilauramidopropyl buylamine, dilauramidopropyl hydroxyethylamine, dilauramidopropyl hydroxypropylamine, distearamidopropyl amine, distearamidopropyl methylamine, dibehenamidopropyl propylamine, dibehenamidopropyl hydroxyethylamine, palmitoamidopropyl trimethyl ammonium chloride, stearamidopropyl trimethylammonium chloride, behenamidopropyl tri hydroxyethalmonium chloride, distearylamidopropyl dimethyl ammonium chloride, dicetylamidodihydroxyethyl ammonium chloride, palmitoylpropylamine, palmitoylpropyl methylamine, palmitoylpropyl diethylamine, palmitoylpropyl dibutylamine, palmitoylpropyl buylamine, palmitoylpropyl dipropylamine, palmitoylpropylpropylamine, palmitoylpropyl dihydroxyethylamine, palmitoylpropyl hydroxyethylamine, palmitoylpropyl dihydroxypropylamine, palmitoylpropyl hydroxypropylamine, myristoylpropyl amine, myristoylpropyl methylamine, myristoylpropyl diethylamine, myristoylpropyl dibutylamine, myristoylpropyl buylamine, myristoylpropyl dipropylamine, myristoylpropyl propylamine, myristoylpropyl dihydroxyethylamine, myristoylpropyl hydroxyethylamine, myristoylpropyl dihydroxypropylamine, myristoylpropyl hydroxypropylamine, stearoylpropyl amine, stearoylpropyl methylamine, stearoylpropyl diethylamine, stearoylpropyl dibutylamine, stearoylpropyl butylamine, stearoylpropyl dipropylamine, behenylpropyl propylamine, behenylpropyl dihydroxyethylamine, behenylpropyl hydroxyethylamine, behenylpropyl dihydroxypropylamine, behenylpropyl hydroxypropylamine, behenylpropyl amine, behenylpropyl methylamine, behenylpropyl diethylamine, behenylpropyl dibutylamine, behenylpropyl butylamine, behenylpropyl dipropylamine, behenylpropyl propylamine, behenylpropyl dihydroxyethylamine, behenylpropyl hydroxyethylamine, behenylpropyl dihydroxypropylamine, behenylpropyl hydroxypropylamine, dipalmitoylpropyl methylamine, dipalmitoylpropyl ethylamine, dipalmitylpropyl butylamine, dipalmitylpropyl propylamine, dipalmitylpropyl hydroxyethylamine, dipalmitylpropyl hydroxypropylamine, dilauroylpropyl amine, dilauroylpropyl methylamine, dilauroylpropyl buylamine, dilauroylpropyl hydroxyethylamine, dilauroylpropyl hydroxypropylamine, distearylpropyl amine, distearylpropyl methylamine, dibehenylpropyl propylamine, dibehenylpropyl hydroxyethylamine, palmitylpropyl trimethyl ammonium chloride, stearylpropyl trimethylammonium chloride, behenylpropyl tri hydroxyethalmonium chloride, distearylpropyl dimethyl ammonium chloride, dicetyldihydroxyethyl ammonium chloride, dioleoylethylhydroxyethylmonium methosulfate, and dicocoylethylhydroxyethylmonium methosulfate.

Cationizable surfactants may be chosen from fatty alkylamines and fatty dialkylamines. In some cases, the fatty dialkylamines may be fatty dimethylamines. Non-limiting examples include dimethyl lauramine, dimethyl behenamine, dimethyl cocamine, dimethyl myristamine, dimethyl palmitamine, dimethyl stearamine, dimethyl tallowamine, dimethyl soyamine, and mixtures thereof.

Fatty dialkylamines include fatty amidoamine compounds, their salts, and mixtures thereof. Non-limiting examples include oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, and palmitamidopropyl dimethylamine.

Non-polymeric, mono-, di-, and/or tri-carboxylic acids may be used to “neutralize” the fatty dialkylamines. In some cases, the one or more non-polymeric, mono-, di-, and/or tri-carboxylic acids include at least one dicarboxylic acid. Non-limiting examples include lactic acid, oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, benzoic acid, and mixtures thereof. In particular, lactic acid or tartaric acid or mixtures thereof are useful, especially in combination with fatty dimethylamines such as, for example, stearamidopropyl dimethylamine.

Nonionic Surfactants

The nonionic surfactant(s) can be, for example, selected from alkyl polyglucosides and alcohols, alpha-diols, alkylphenols and esters of fatty acids, being ethoxylated, propoxylated or glycerolated and having at least one fatty chain comprising, for example, from 8 to 18 carbon atoms, it being possible for the number of ethylene oxide or propylene oxide groups to range from 2 to 50, and for the number of glycerol groups to range from 1 to 30. Maltose derivatives may also be mentioned.

Non-limiting mention may also be made of copolymers of ethylene oxide and/or of propylene oxide; condensates of ethylene oxide and/or of propylene oxide with fatty alcohols; polyethoxylated fatty amides comprising, for example, from 2 to 30 mol of ethylene oxide; polyglycerolated fatty amides comprising, for example, from 1.5 to 5 glycerol groups, such as from 1.5 to 4; ethoxylated fatty acid esters of sorbitan comprising from 2 to 30 mol of ethylene oxide; ethoxylated oils from plant origin; fatty acid esters of sucrose; fatty acid esters of polyethylene glycol; polyethoxylated fatty acid mono or diesters of glycerol (C₆-C₂₄)alkylpolyglycosides; N-(C₆-C₂₄)alkylglucamine derivatives, amine oxides such as (C₁₀-C₁₄)alkylamine oxides or N-(C₁₀-C₁₄)acylaminopropylmorpholine oxides; and mixtures thereof.

Such nonionic surfactants may preferably be chosen from polyoxyalkylenated or polyglycerolated nonionic surfactants. The oxyalkylene units are more particularly oxyethylene or oxypropylene units, or a combination thereof, and are preferably oxyethylene units.

In some cases, the nonionic surfactant may be selected from esters of polyols with fatty acids with a saturated or unsaturated chain containing for example from 8 to 24 carbon atoms, preferably 12 to 22 carbon atoms, and alkoxylated derivatives thereof, preferably with a number of alkyleneoxide of from 10 to 200, and more preferably from 10 to 100, such as glyceryl esters of a C₈-C₂₄, preferably C₁₂-C₂₂, fatty acid or acids and alkoxylated derivatives thereof, preferably with a number of alkyleneoxide of from 10 to 200, and more preferably from 10 to 100; polyethylene glycol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sorbitol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sugar (sucrose, glucose, alkylglycose) esters of a C₈-C₂₄, preferably C₁₂-C₂₂, fatty acid or acids and alkoxylated derivatives thereof, preferably with a number of alkyleneoxide of from 10 to 200, and more preferably from 10 to 100; ethers of fatty alcohols; ethers of sugar and a C₈-C₂₄, preferably C₁₂-C₂₂, fatty alcohol or alcohols; and mixtures thereof.

Examples of ethoxylated fatty esters that may be mentioned include the adducts of ethylene oxide with esters of lauric acid, palmitic acid, stearic acid or behenic acid, and mixtures thereof, especially those containing from 9 to 100 oxyethylene groups, such as PEG-9 to PEG-50 laurate (as the CTFA names: PEG-9 laurate to PEG-50 laurate); PEG-9 to PEG-50 palmitate (as the CTFA names: PEG-9 palmitate to PEG-50 palmitate); PEG-9 to PEG-50 stearate (as the CTFA names: PEG-9 stearate to PEG-50 stearate); PEG-9 to PEG-50 palmitostearate; PEG-9 to PEG-50 behenate (as the CTFA names: PEG-9 behenate to PEG-50 behenate); polyethylene glycol 100 EO monostearate (CTFA name: PEG-100 stearate); and mixtures thereof.

As glyceryl esters of fatty acids, glyceryl stearate (glyceryl mono-, di- and/or tristearate) (CTFA name: glyceryl stearate) or glyceryl ricinoleate and mixtures thereof can in particular be cited.

As glyceryl esters of C₈-C₂₄ alkoxylated fatty acids, polyethoxylated glyceryl stearate (glyceryl mono-, di- and/or tristearate) such as PEG-20 glyceryl stearate can for example be cited.

Mixtures of these surfactants, such as for example the product containing glyceryl stearate and PEG-100 stearate, marketed under the name ARLACEL 165 by Uniqema, and the product containing glyceryl stearate (glyceryl mono- and distearate) and potassium stearate marketed under the name TEG1N by Goldschmidt (CTFA name: glyceryl stearate SE), can also be used.

Alkyl polyglucosides are a class of nonionic surfactants. Useful polyglucosides include alkyl polyglucosides having the following formula (IX):

R¹—O—(R²O)ₙ—Z(x) (XII)

wherein R¹ is an alkyl group having 8-18 carbon atoms;

R² is an ethylene or propylene group;

Z is a saccharide group with 5 to 6 carbon atoms;

n is an integer from 0 to 10; and

x is an integer from 1 to 5.

Useful alkyl poly glucosides include lauryl glucoside, octyl glucoside, decyl glucoside, coca glucoside, sucrose laurate, caprylyl/capryl glucoside, and sodium lauryl glucose carboxylate, and mixtures thereof. Typically, the at least one alkyl poly glucoside compound is selected from the group consisting of lauryl glucoside, decyl glucoside and coca glucoside, and more typically lauryl glucoside. In some instances, decyl glucoside is particularly preferred.

Amphoteric Surfactants

Useful amphoteric surfactants include betaines, alkyl sultaines, alkyl amphoacetates, alkyl amphoproprionates, and mixtures thereof. Non-limiting examples of useful amphoteric surfactants are provided below.

Betaines

Useful betaines include those of the following formulae (XIIIa-XIIId):

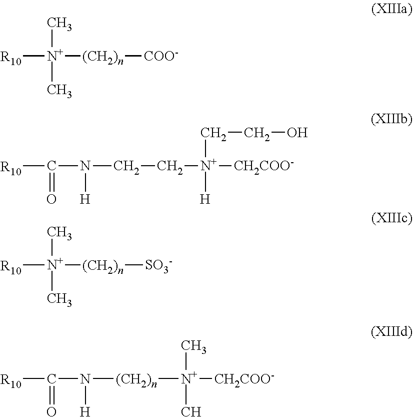

wherein R₁₀ is an alkyl group having 8-18 carbon atoms; and n is an integer from 1 to 3.

Particularly useful betaines include, for example, coca betaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, and mixtures thereof. Typically, at least one betaine compound is selected from coco betaine, behenyl betaine, capryl/capramidopropyl betaine, and lauryl betaine, and mixtures thereof. Particularly preferred betaines include coco betaine and cocamidopropyl betaine.

The total amount of betaines in the hair treatment composition, if present, may vary but is typically from about 0.1 to about 10 wt. %, based on the total weight of the hair treatment composition. In some instance, the total amount of betaines(s) in the hair treatment composition is from 0.1 to about 5 wt. %, about 0.1 to about 3 wt. %, about 1 to about 10 wt. %, about 1 to about 5 wt. %, or about 1 to about 3 wt. %, including all ranges and subranges therebetween.

Alkyl Sulltaines

Non-limiting examples of alkyl sultaines include hydroxyl sultaines of formula

(XIV)

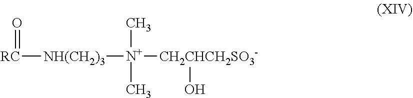

wherein R is an alkyl group having 8-18 carbon atoms. More specific examples include, but are not limited to cocamidopropyl hydroxysultaine, lauryl hydroxysultaine, and a mixture thereof.

Alkyl Amphoacetates and Alkyl Amphodiacetates

Useful alkyl amphoacetates and alkyl amphodiacetates include those of Formula (XV) and (XVI):

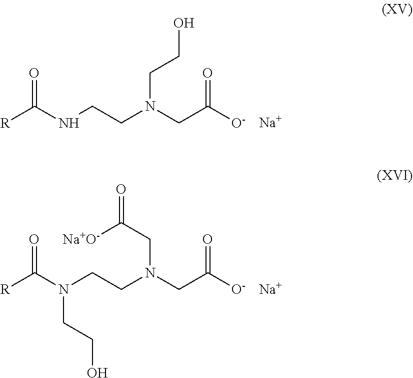

wherein R is an alkyl group having 8-18 carbon atoms. Sodium is shown as the cation in the above formulae but the cation may be an alkali metal ion such as sodium or potassium, ammonium ions, or alkanolammonium ions such as monoethanolammonium or triethanolammonium ions. A more specific, but non-limiting example, is sodium lauroamphoacetate.

Alkyl Amphopropionates

Non-limiting examples of amphopropionates include cocoamphopropionate, caprylamphopropionate, cornamphopropionate, caproamphopropionate, oleoamphopropionate, isostearoamphopropionate, stearoamphopropionate, lauroamphopropionate, salts thereof, and a mixture thereof.

Fatty Compounds

Suitable fatty compounds, if present, include or may be chosen from oils, mineral oil, alkanes (paraffins), fatty alcohols, fatty acids, fatty alcohol derivatives, fatty acid derivatives, esters of fatty alcohols, hydroxy-substituted fatty acids, waxes, triglyceride compounds, lanolin, and a mixture thereof. Preferably, the fatty compound comprises a triglyceride, such as C₁₀-C₁₈triglycerides, caprylic/capric triglycerides, or a mixture thereof. In some instances, the fatty compound comprises at least two of triglyceride, isononyl isononanoate, and glycine soja (soybean) oil/glycine soja oil.

Fatty Alcohols

Suitable fatty alcohols, if present, include those having a fatty group with a carbon chain of greater than 8 carbon atoms, 8 to 50 carbon atoms, 8 to 40 carbon atoms, 8 to 30 carbon atoms, 8 to 22 carbon atoms, 12 to 22 carbon atoms, or 12 to 18 carbon atoms, including all ranges and subranges therebetween. In some instances, the fatty group of the fatty alcohols has a carbon chain of 10 to 20 carbon atoms or 10 to 18 carbon atoms. The fatty alcohols may be chosen from polyethylene glycol ethers, such as those having a fatty alcohol group with a carbon chain of 12 to 16 or 12 to 14 carbon atoms.

The fatty alcohol portion is preferably hydrogenated (for example, stearyl, lauryl, cetyl, cetearyl); however, the fatty alcohol may contain one or more double bonds (for example, oleyl). Non-limiting examples of fatty alcohols include decyl alcohol, undecyl alcohol, dodecyl alcohol, myristyl alcohol, lauryl alcohol, cetyl alcohol, stearyl alcohol, cetearyl alcohol (cetyl alcohol and stearyl alcohol), isostearyl alcohol, isocetyl alcohol, behenyl alcohol, linalool, oleyl alcohol, cis-4-t-butylcyclohexanol, isotridecyl alcohol, myricyl alcohol, and a mixture thereof. In some cases, the fatty alcohols comprise at least one of or may be chosen from myristyl alcohol, lauryl alcohol, cetyl alcohol, stearyl alcohol, cetearyl alcohol, isostearyl alcohol, oleyl alcohol, isotridecyl alcohol, and a mixture thereof.

The fatty alcohol may be saturated or unsaturated. Exemplary saturated liquid fatty alcohols may be branched and optionally contain in their structure at least one aromatic or non-aromatic ring. In some instances, however, the fatty alcohols are acyclic. Non-limiting examples of liquid saturated fatty alcohols include octyldodecanol, isostearyl alcohol, and 2-hexyldecanol.

Exemplary unsaturated liquid fatty alcohol may include in their structure at least one double or triple bond. For example, the fatty alcohols may include several double bonds (such as 2 or 3 double bond), which may be conjugated or non-conjugated. The unsaturated fatty alcohols can be linear or branched and may be acyclic or include in their structure at least one aromatic or non-aromatic ring. Liquid unsaturated fatty alcohols may include or be chosen from oleyl alcohol, linoleyl alcohol, linolenyl alcohol and undecylenyl alcohol.

The fatty alcohols may be alkoxylated fatty alcohols, e.g., having about 1 to about 100 moles of an alkylene oxide per mole of alkoxylated fatty alcohol. For example, the alkoxylated fatty alcohols may be alkoxylated with about 1 to about 80 moles, about 2 to about 50, about 5 to about 45 moles, about 10 to about 40 moles, or 15 to about 35 mores, including all ranges and subranges therebetween, of an alkylene oxide per mole of alkoxylated fatty alcohol.

As examples of alkoxylated fatty alcohols, steareth (for example, steareth-2, steareth-20, and steareth-21), laureth (for example, laureth-4, and laureth-12), ceteth (for example, ceteth-10 and ceteth-20) and ceteareth (for example, ceteareth-2, ceteareth-10, and ceteareth-20) are mentioned. In at least one instance, the one or more alkoxylated fatty alcohols include steareth-20. In some instances, the one or more alkoxylated fatty alcohols may be exclusively steareth-20.

Additional fatty alcohol derivatives that may, optionally be suitable include methyl stearyl ether; 2-ethylhexyl dodecyl ether; stearyl acetate; cetyl propionate; the ceteth series of compounds, such as ceteth-1 through ceteth-45, which are ethylene glycol ethers of cetyl alcohol, wherein the numeric designation indicates the number of ethylene glycol moieties present; the steareth series of compounds such as steareth-1 through 10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e. a mixture of fatty alcohols containing predominantly cetyl and stearyl alcohol, wherein the numeric designation indicates the number of ethylene glycol moieties present; C1-C30 alkyl ethers of the ceteth, steareth, and ceteareth compounds just described; polyoxyethylene ethers of branched alcohols such as octyldodecyl alcohol, dodecylpentadecyl alcohol, hexyldecyl alcohol, and isostearyl alcohol; polyoxyethylene ethers of behenyl alcohol; PPG ethers such as PPG-9-steareth-3, PPG-11 stearyl ether, PPG8-ceteth-1, and PPG-10 cetyl ether; and a mixture thereof.

Fatty Acids

In some instances, the fatty compounds may be chosen from fatty acids, fatty acid derivatives, esters of fatty acids, hydroxyl-substituted fatty acids, and alkoxylated fatty acids. The fatty acids may be straight or branched chain acids and/or may be saturated or unsaturated. Non-limiting examples of fatty acids include diacids, triacids, and other multiple acids as well as salts of these fatty acids. For example, the fatty acid may optionally include or be chosen from lauric acid, palmitic acid, stearic acid, behenic acid, arichidonic acid, oleic acid, isostearic acid, sebacic acid, and a mixture thereof. In some cases, the fatty acids are selected from the group consisting of palmitic acid, stearic acid, and a mixture thereof.

Non-limiting examples of polyglycerol esters of fatty acids include those of the following formula:

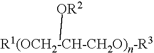

wherein the average value of n is about 3 and R¹, R² and R³ each may independently be a fatty acid moiety or hydrogen, provided that at least one of R¹, R², and R³ is a fatty acid moiety. For instance, R¹, R² and R³ may be saturated or unsaturated, straight or branched, and have a length of C₁-C₄₀, C₁-C₃₀, C₁-C₂₅, or C₁-C₂₀, C₁-C₁₆, or C₁-C_{10.}

The fatty acid derivatives are defined herein to include fatty acid esters of the fatty alcohols as defined above, fatty acid esters of the fatty alcohol derivatives as defined above when such fatty alcohol derivatives have an esterifiable hydroxyl group, fatty acid esters of alcohols other than the fatty alcohols and the fatty alcohol derivatives described above, hydroxy-substituted fatty acids, and a mixture thereof. Non-limiting examples of fatty acid derivatives include ricinoleic acid, glycerol monostearate, 12-hydroxy stearic acid, ethyl stearate, cetyl stearate, cetyl palmitate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, dimethyl sebacate, PEG-15 cocoate, PPG-15 stearate, glyceryl monostearate, glyceryl distearate, glyceryl tristearate, PEG-8 laurate, PPG-2 isostearate, PPG-9 laurate, and a mixture thereof. Preferred for use herein are glycerol monostearate, 12-hydroxy stearic acid, and a mixture thereof.

Waxes

The fatty compounds may, in some instances, include or be chosen from one or more waxes. Non-limiting examples of waxes in this category include for example, synthetic wax, ceresin, paraffin, ozokerite, polyethylene waxes, illipe butter, beeswax, carnauba, microcrystalline, lanolin, lanolin derivatives, candelilla, cocoa butter, shellac wax, spermaceti, bran wax, capok wax, sugar cane wax, montan wax, whale wax, bayberry wax, acacia decurrents flower wax, vegetable waxes (such as sunflower seed (helianthus annuus), carnauba, candelilla, ouricury or japan wax or cork fibre or sugarcane waxes), or a mixture thereof.

Oils

In some instances, the fatty compounds may include or be chosen from one or more oil(s). Suitable oils include, but are not limited to, natural oils, such as coconut oil; hydrocarbons, such as mineral oil and hydrogenated polyisobutene; fatty alcohols, such as octyldodecanol; esters, such as C₁₂-C₁₅ alkyl benzoate; diesters, such as propylene dipelarganate; and triesters, such as glyceryl trioctanoate. Non-limiting examples of oils that may, optionally, be included in the hair treatment compositions include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, or combinations of octyldodecanol, acetylated lanolin alcohol, cetyl acetate, isododecanol, polyglyceryl-3-diisostearate, castor oil, lanolin and lanolin derivatives, triisocetyl citrate, sorbitan sesquioleate, C₁₀-C₁₈triglycerides, caprylic/capric/triglycerides, coconut oil, corn oil, cottonseed oil, glyceryl triacetyl hydroxystearate, glyceryl triacetyl ricinoleate, glyceryl trioctanoate, hydrogenated castor oil, linseed oil, mink oil, olive oil, palm oil, illipe butter, rapeseed oil, soybean oil, sunflower seed oil, tallow, tricaprin, trihydroxystearin, triisostearin, trilaurin, trilinolein, trimyristin, triolein, tripalmitin, tristearin, walnut oil, wheat germ oil, cholesterol, or combinations thereof.

Implementation of the present disclosure is provided by way of the following examples. The examples serve to illustrate the technology without being limiting in nature.

### EXAMPLES

The following Examples are intended to be non-restrictive and explanatory only.

The ingredient amounts in the compositions/formulas described below are expressed in % by weight, based on the total weight of the composition.

### Example 1

### (Inventive Compositions)

| | | | Inventive | | |
|---|---|---|---|---|---|
| | | | A | B | C |
| | | | Shampoo | Shampoo | Conditioner |
| (a) | Ascorbyl Glucoside | ASCORBYL GLUCOSIDE | 1.0 | 0.5 | 0.5 |
| (b) | Amino Acid Chelant | GLYCINE | 0.15 | 0.15 | 0.15 |
| (c) | Glutamate Chelant | TETRASODIUM GLUTAMATE | 0.15 | 0.15 | 0.15 |
| | | DIACETATE | | | |
| (d) | Anionic Surfactant | SODIUM LAURETH SULFATE | 10.5 | 10.5 | |
| | Cationic Surfactant | CETRIMONIUM CHLORIDE, | | | 1.8 |
| | | BEHENTRIMONIUM | | | |
| | | METHOSULFATE, AND | | | |
| | | BEHENTRIMONIUM | | | |
| | | CHLORIDE | | | |
| | Amphoteric Surfactant | COCO-BETAINE | 1.8 | 1.8 | |
| | Nonionic Surfactant | TRIDECETH-6 | | | 0.1 |
| (f) | Water Soluble Solvent | HEXYLENE GLYCOL, | 2.5 | 2.5 | 1.2 |
| | | GLYCERIN, AND | | | |
| | | ISOPROPYL ALCOHOL | | | |
| (g) | Cationic Conditioning | HYDROXYPROPYL GUAR | 0.3 | 0.3 | |
| | Polymer | HYDROXYPROPYL- | | | |
| | | TRIMONIUM CHLORIDE | | | |
| | Fatty Compound | CETEARYL ALCOHOL | | | 4.0 |
| | Silicone | AMODIMETHICONE | | | 0.9 |
| | Miscellaneous | SALTS, PH ADJUSTERS, | 0-4 | 0-4 | 0-4 |
| | | PRESERVATIVES, | | | |
| | | PERFUME, ETC. | | | |
| (e) | | WATER | QS 100 | QS 100 | QS 100 |
| | pH | PH | 5.0-5.6 | 5.0-5.6 | 3.5-4.5 |

### Example 2

### (Inventive and Comparative Shampoo Compositions)

| | | | Inventive | Comparative | | |
|---|---|---|---|---|---|---|
| | | | B | B-1 | B-2 | B-3 |
| | | | Shampoo | Shampoo | Shampoo | Shampoo |
| (a) | Ascorbyl | ASCORBYL GLUCOSIDE | 0.5 | — | 0.5 | — |
| | Glucoside | | | | | |
| (b) | Amino Acid | GLYCINE | 0.15 | 0.15 | — | — |
| | Chelant | | | | | |
| (c) | Glutamate | TETRASODIUM | 0.15 | 0.15 | — | — |
| | Chelant | GLUTAMATE DIACETATE | | | | |
| (d) | Anionic | SODIUM LAURETH | 10.5 | 10.5 | 10.5 | 10.5 |
| | Surfactant | SULFATE | | | | |
| | Amphoteric | COCO-BETAINE | 1.8 | 1.8 | 1.8 | 1.8 |
| | Surfactant | | | | | |
| (f) | Water Soluble | HEXYLENE GLYCOL, | 2.5 | 2.5 | 2.5 | 2.5 |
| | Solvent | GLYCERIN, AND | | | | |
| | | ISOPROPYL ALCOHOL | | | | |
| (g) | Cationic | HYDROXYPROPYL GUAR | 0.3 | 0.3 | 0.3 | 0.3 |
| | Conditioning | HYDROXYPROPYL- | | | | |
| | Polymer | TRIMONIUM CHLORIDE | | | | |
| | Miscellaneous | SALTS, PH | 0-4 | 0-4 | 0-4 | 0-4 |
| | | ADJUSTERS, | | | | |
| | | PRESERVATIVES, | | | | |
| | | PERFUME, ETC. | | | | |
| (e) | | WATER | QS 100 | QS 100 | QS 100 | QS 100 |
| | pH | pH | 5.0-5.6 | 5.0-5.6 | 5.0-5.6 | 5.0-5.6 |

### Example 3

### (Inventive and Comparative Conditioner Compositions)

| | | | C | C-1 | C-2 | C-3 |
|---|---|---|---|---|---|---|
| | | | Conditioner | Conditioner | Conditioner | Conditioner |
| (a) | Ascorbyl | ASCORBYL | 0.5 | — | 0.5 | — |
| | Glucoside | GLUCOSIDE | | | | |
| (b) | Amino Acid | GLYCINE | 0.15 | 0.15 | — | — |
| | Chelant | | | | | |
| (c) | Glutamate | TETRASODIUM | 0.15 | 0.15 | — | — |
| | Chelant | GLUTAMATE | | | | |
| | | DIACETATE | | | | |
| (d) | Cationic | CETRIMONIUM | 1.8 | 1.8 | 1.8 | 1.8 |
| | Surfactant | CHLORIDE, | | | | |
| | | BEHENTRIMONIUM | | | | |
| | | METHOSULFATE, AND | | | | |
| | | BEHENTRIMONIUM | | | | |
| | | CHLORIDE | | | | |
| | Nonionic | TRIDECETH-6 | 0-1 | 0-1 | 0-1 | 0-1 |
| | Surfactant | | | | | |
| (f) | Water Soluble | HEXYLENE GLYCOL, | 1.2 | 1.2 | 1.2 | 1.2 |
| | Solvent | GLYCERIN, AND | | | | |
| | | ISOPROPYL ALCOHOL | | | | |
| | Fatty | CETEARYL ALCOHOL | 4.0 | 4.0 | 4.0 | 4.0 |
| | Compound | | | | | |
| | Silicone | AMODIMETHICONE | 0.9 | 0.9 | 0.9 | 0.9 |
| (h) | Miscellaneous | SALTS, PH | 0-4 | 0-4 | 0-4 | 0-4 |
| | | ADJUSTERS, | | | | |
| | | PRESERVATIVES, | | | | |
| | | PERFUME, ETC. | | | | |
| (e) | | WATER | QS 100 | QS 100 | QS 100 | QS100 |
| | pH | pH | 3.5-4.5 | 3.5-4.5 | 3.5-4.5 | 3.5-4.5 |

Example 4 (Brightening Study)

A brightening study was carried out to determine how the inventive compositions of the instant case and comparative compositions impact the brightness and shine of hair. Highly bleached Caucasian hair swatches (SA40, 2.7 g, 27 cm) were soaked in a 2% aqueous solution of copper sulfate overnight. The next day, the hair swatches were rinsed with water and allowed to air dry. After drying, the hair swatches were shampooed with one of the shampoos from Example 2 (B, B-1, B-2, and B-3). The shampoo was massaged throughout the hair for 5 minutes and then rinsed from the hair swatches with water. After removing excess water, the hair swatches were conditioned with one of the conditioners from Example 3 (C, C-1, C-2, and C-3). The conditioner was massaged throughout the hair for 5 minutes and then rinsed from the hair swatches with water. The shampooing and conditioning were repeated for a total of 7 treatments each. After the seventh treatment, the hair swatches were dried with a blow dryer. The brightness and shine of the hair swatches was visually evaluated by a panel of experts relative to a control and the results (averages) reported in the table below. The control consisted of highly bleached Caucasian hair swatches (SA40, 2.7 g, 27 cm) that were soaked in the 2% aqueous solution of copper sulfate overnight but not subsequently treated with a shampoo and conditioner.

| | Inventive | Comparative | | |
|---|---|---|---|---|
| | Inventive | Comparative | Comparative | Comparative |
| | Shampoo B/ | Shampoo B-1/ | Shampoo B-2/ | Shampoo B-3/ |
| | Inventive | Comparative | Comparative | Comparative |
| Main Constituents | Conditioner C | Conditioner C-1 | Conditioner C-2 | Conditioner C-3 |
| Ascorbyl | ✓ | | ✓ | |
| Glucoside | | | | |
| Chelators | ✓ | ✓ | | |
| (Glycine and | | | | |
| GLDA) | | | | |

Attributes

| | | | | |
|---|---|---|---|---|
| Brightening | +++ | ++ | ++ | + |
| Shine | +++ | + | + | = |

“+++” indicates that the hair swatches were much brighter/much shinier than the control
“++” indicates that the hair swatches were brighter/shinier than the control
“+” indicates that the hair swatches were somewhat brighter/somewhat shinier than the control
“=” indicates that the hair swatches were did not differ from the control with respect to brightness/shininess

The data show that ascorbyl glucoside provides some improvement with respect to brightening and slight improvement with respect to shine. Similarly, the chelants provided some improvement with respect to brightening and slight improvement with respect to shine. The use of both ascorbyl glucoside and chelants (glycine and GLDA), however, provided a significant and surprising improvement with respect to both brightness and shine.

The foregoing description illustrates and describes the invention. The disclosure shows and describes only the preferred embodiments but it should be understood that the invention is capable to use in various other combinations, modifications, and environments and is capable of changes or modifications within the scope of the inventive concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the claimed invention.

The terms “a,” “an,” and “the” are understood to encompass the plural as well as the singular.

Thus, the term “a mixture thereof” also relates to “mixtures thereof.” Throughout the disclosure, if the term “a mixture thereof” is used, following a list of elements as shown in the following example where letters A-F represent the elements: “one or more elements selected from the group consisting of A, B, C, D, E, F, or mixtures thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements chosen from A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of

E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting.

The expression “one or more” means “at least one” and thus includes individual components as well as mixtures/combinations.

The term “plurality” means “more than one” or “two or more.”

Some of the various categories of components identified for the hair-treatment compositions may overlap. In such cases where overlap may exist and the composition/product includes two overlapping components (or more than two overlapping components), an overlapping component does not represent more than one component. For example, a fatty acid may be defined as both a “fatty compound” and a “surfactant/emulsifier.” If a particular composition/product includes both a fatty compound component and an emulsifier component, a single fatty acid can serve as only a fatty compound or a surfactant/emulsifier (a single fatty acid does not serve as both the fatty compound and the surfactant/emulsifier).

All percentages, parts and ratios herein are based upon the total weight of the compositions of the present invention, unless otherwise indicated.

All ranges and values disclosed herein are inclusive and combinable. For examples, any value or point described herein that falls within a range described herein can serve as a minimum or maximum value to derive a sub-range, etc. Furthermore, all ranges provided are meant to include every specific range within, and combination of sub-ranges between, the given ranges. Thus, a range from 1-5, includes specifically points 1, 2, 3, 4 and 5, as well as sub-ranges such as 2-5, 3-5, 2-3, 2-4, 1-4, etc.; and points of 1, 2, 3, 4, and 5 includes ranges and sub-ranges of 1-5, 2-5, 3-5, 2-3, 2-4, 1-4, etc.

Other than in the operating examples, or where otherwise indicated, all numbers expressing quantities of ingredients and/or reaction conditions are understood to be modified by “about,” whether or not expressly stated. Additionally, all numbers are intended to represent exact figures as additional embodiments, whether or not modified by “about.” For example, “an amount of about 1%” includes an amount of exactly 1%.

As a further example, “an amount of 1%” includes an amount of about 1%. The term “about” is generally understood to encompass a range of +/−10% from the stated number, and is intended to cover amounts of +/−1%, 2%, 3%, 4%, 5%, 6%, 7%, 8%, 9%, and 10%.

The term “surfactants” includes salts of the surfactants even if not explicitly stated. In other words, whenever the disclosure refers to a surfactant, it is intended that salts of the surfactant are also encompassed to the extent such salts exist, even though the specification may not specifically refer to a salt (or may not refer to a salt in every instance throughout the disclosure), for example, by using language such as “a salt thereof” or “salts thereof.” Sodium and potassium are common cations that form salts with surfactants. However, additional cations such as ammonium ions, or alkanolammonium ions such as monoethanolammonium or triethanolammonium ions, may also form salts of surfactants.

The term “substantially free” or “essentially free” as used herein means the specific material may be present in small amounts that do not materially affect the basic and novel characteristics of the claimed invention. For instance, there may be less than 2% by weight of a specific material added to a composition, based on the total weight of the compositions (provided that an amount of less than 2% by weight does not materially affect the basic and novel characteristics of the claimed invention. Similarly, the compositions may include less than 2 wt %, less than 1.5 wt %, less than 1 wt %, less than 0.5 wt %, less than 0.1 wt %, less than 0.05 wt %, or less than 0.01 wt %, or none of the specified material. The term “substantially free” or “essentially free” as used herein may also mean that the specific material is not added to the composition but may still be present in a raw material that is included in the composition.

Furthermore, all components that are positively set forth in the instant disclosure may be negatively excluded from the claims, e.g., a claimed composition may be “free,” “essentially free” (or “substantially free”) of one or more components that are positively set forth in the instant disclosure.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A hair treatment composition comprising:
(a) ascorbyl glucoside;
(b) an amino acid chelant;
(c) N,N-dicarboxymethyl glutamic acid, a salt thereof, and/or a combination thereof;
(d) one or more surfactants; and
(e) water.

2. The hair treatment composition of claim 1 comprising;
(a) about 0.1 to about 5 wt. % of the ascorbyl glucoside;
(b) about 0.01 to about 3 wt. % of the amino acid chelant;
(c) about 0.01 to about 3 wt. % of the N,N-dicarboxymethyl glutamic acid, a salt thereof, and/or a combination thereof;
(d) about 0.5 to about 25 wt. % of the one or more surfactants; and
(e) about 50 to about 95 wt. % of the water;
wherein all weight percentages are based on the total weight of the hair treatment composition.

3. The hair treatment composition of claim 1, wherein the amino acid chelant is chosen from glycine, lysine, methionine, and combinations thereof.

4. The hair treatment composition of claim 3, wherein the amino acid chelant is glycine.

5. The hair treatment composition of claim 1, wherein the N,N-dicarboxymethyl glutamic acid, a salt thereof, and/or a combination thereof is tetrasodium glutamate diacetate.

6. The hair treatment composition of claim 1, further comprising:
(f) about 0.1 to about 25 wt. % of one or more water-soluble solvents.

7. The hair treatment composition of claim 6, wherein the water-soluble solvents are chosen from glycerin, mono-alcohols, polyols (polyhydric alcohols), glycols, and a mixture thereof.

8. The hair treatment composition of claim 1 being a shampoo, wherein the one or more surfactants (d) comprises one or more anionic surfactants, wherein the one or more anionic surfactants are in an amount of about 5 to about 25 wt. %, based on the total weight of the hair treatment composition.

9. The hair treatment composition of claim 8, wherein the one or more anionic surfactants are chosen from alkyl sulfates, alkyl ether sulfates, alkyl sulfonates, alkyl sulfosuccinates, alkyl sulfoacetates, acyl isethionates, alkoxylated monoacids, acyl amino acids such as acyl taurates, acyl glycinates, acyl glutamates, acyl sarcosinates, salts thereof, and a mixture thereof.

10. The hair treatment composition of claim 1 being a conditioner, wherein the one or more surfactants (d) comprises one or more cationic surfactants, wherein the one or more cationic surfactants are in an amount of about 0.1 to about 10 wt. %, based on the total weight of the hair treatment composition.

11. The hair treatment composition of claim 10, wherein the one or more cationic surfactants are chosen from cetrimonium chloride, stearimonium chloride, behentrimonium chloride, behentrimonium methosulfate, behenam idopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, and mixtures thereof.

12. The hair treatment composition of claim 1, further comprising:
(g) one or more cationic conditioning polymers.

13. The hair treatment composition of claim 12, wherein the one or more cationic conditioning polymers are chosen from cationic cellulose derivatives, quaternized hydroxyethyl cellulose, cationic starch derivatives, cationic guar gum derivatives (hydroxypropyl guar hydroxypropyltrimonium chloride), copolymers of acrylamide and dimethyldiallyammonium chloride, polyquaterniums, and a mixture thereof.

14. The hair treatment composition of claim 1 being free from hair coloring dyes, pigments, and colorants.

15. The hair treatment composition of claim 1 that is not a hair coloring or bleaching composition and not a permanent hair shaping composition.

16. The hair treatment composition of claim 1 being a shampoo comprising:
(a) about 0.1 to about 5 wt. % of the ascorbyl glucoside;
(b) about 0.01 to about 3 wt. % of an amino acid chelant, which is glycine;
(c) about 0.01 to about 3 wt. % of the N,N-dicarboxymethyl glutamic acid, a salt thereof, and/or a combination thereof;
(d) about 5 to about 25 wt. % the one or more anionic surfactants;
(e) about 50 to about 95 wt. % of the water; and
(f) about 0.1 to about 25 wt. % of one or more water-soluble solvents.
wherein all weight percentages are based on the total weight of the hair treatment composition.

17. The hair treatment composition of claim 1 being a conditioner comprising:
(a) about 0.1 to about 5 wt. % of the ascorbyl glucoside;
(b) about 0.01 to about 3 wt. % of an amino acid chelant, which is glycine;
(c) about 0.01 to about 3 wt. % of the N,N-dicarboxymethyl glutamic acid, a salt thereof, and/or a combination thereof;
(d) about 0.1 to about 10 wt. % the one or more cationic surfactants;
(e) about 50 to about 95 wt. % of the water;
(f) about 0.1 to about 25 wt. % of one or more water-soluble solvents;
(g) about 0.1 to about 25 wt. % of one or more fatty compounds; and
(g) optionally, one or more silicones;
wherein all weight percentages are based on the total weight of the hair treatment composition.

18. A kit comprising:
(i) a shampoo composition comprising:
(a) ascorbyl glucoside;
(b) an amino acid chelant;
(c) N,N-dicarboxymethyl glutamic acid, a salt thereof, and/or a combination thereof;
(d) one or more anionic surfactants; and
(e) water; and/or
(ii) a conditioner composition comprising:
(a) ascorbyl glucoside;
(b) an amino acid chelant;
(c) N,N-dicarboxymethyl glutamic acid, a salt thereof, and/or a combination thereof;
(d) one or more cationic surfactants; and
(e) water.

19. A method for treating hair comprising:
(i) applying a hair treatment composition to the hair, wherein the hair treatment composition comprises:
(a) ascorbyl glucoside;
(b) an amino acid chelant;
(c) N,N-dicarboxymethyl glutamic acid, a salt thereof, and/or a combination thereof;
(d) one or more surfactants; and
(e) water; and
(ii) rinsing the hair treatment composition from the hair.

20. The method of claim 19, wherein the hair treatment composition is a shampoo or a conditioner and/or wherein the hair has been artificially colored, bleached, or chemically treated.
